(19) United States
(12) Patent Application Publication
(10) Pub. No.: US 2015/0297310 A1
(43) Pub. Date: Oct. 22, 2015

(54) METHODS FOR SELECTING A TREATMENT FOR CANCER

(71) Applicant: Georgia Regents Research Institute, Inc., Augusta, GA (US)

(72) Inventors: Samir N. Khleif, Silver Spring, MD (US); Mikayel Mkrtichyan, Augusta, GA (US)

(21) Appl. No.: 14/689,314

(22) Filed: Apr. 17, 2015

Related U.S. Application Data
(60) Provisional application No. 61/980,790, filed on Apr. 17, 2014.

(51) Int. Cl. A61B 19/00; A61N 5/06; A61B 5/145; A61N 5/10

(52) U.S. Cl. CPC A61B 19/52; A61N 5/10; A61N 5/062; A61B 5/14546

(57) ABSTRACT

Biomarkers predictive of a subject's likelihood of responding to an immunotherapy including blockade of PD-1 inhibitory signaling, and method of use thereof are disclosed. The biomarkers are HIF1-alpha, KDR, CXCL13, and IL7R. Exemplary methods include identifying or selecting a subject who may benefit from treatment with an immunotherapy including blockade of PD-1 signaling, and methods of predicting responsiveness of a subject suffering from cancer to treatment with an immunotherapy including blockade of PD-1 signaling. Preferred immunotherapies and methods of treating subjects with cancer are also provided.

### CROSS-REFERENCE TO RELATED APPLICATIONS

This application claims benefit of and priority to U.S. Provisional Application No. 61/980,790, filed on Apr. 17, 2014, and which is incorporated by reference in its entity.

### REFERENCE TO SEQUENCE LISTING

The Sequence Listing submitted Apr. 17, 2015 as a text file named “GRU_{—}2014-019_ST25.txt,” created on Apr. 17, 2015, and having a size of 54,000 bytes is hereby incorporated by reference pursuant to 37 C.F.R. §1.52(e)(5).

### FIELD OF THE INVENTION

This invention relates to biomarkers for determining the efficacy of cancer immunotherapies including blockade of PD-1 mediated signaling, and methods of selecting subjects and treatments based on analysis of levels expression of the biomarkers in a cancer sample.

### BACKGROUND OF THE INVENTION

Immunotherapy refers to the treatment of disease by inducing, enhancing, or suppressing an immune response. Immunotherapies designed to elicit or amplify an immune response are classified as activation immunotherapies, and are being developed as treatments for cancer wherein the immunotherapy stimulates the immune system to reject and destroy cancer cells. Initial immunotherapy treatments involved administration of cytokines such as interleukin. Thereafter the adverse effects of such intravenously administered cytokines lead to the extraction of the lymphocytes from the blood and expanding in vitro against tumor antigen before injecting the cells with appropriate stimulatory cytokines. The cells will then specifically target and destroy the tumor expressing antigen against which they have been raised.

More recent attempts at immunotherapy include increasing an immune response to cancer cells by blocking an immune inhibitory pathway. One such pathway is the PD-1 inhibitory signaling pathway. The primary result of PD-1 ligation by its endogenous ligands is to inhibit signaling downstream of the T cell Receptor (TCR). Therefore, signal transduction via PD-1 usually provides a suppressive or inhibitory signal to the T cell that results in decreased T cell proliferation or other reduction in T cell activation.

Compositions and methods for increasing T cell responses, for example, to an antigen such as a cancer antigen, by administering the subject a compound that reduces PD-1 inhibitory signal transduction in immune cells, especially T cells have been developed. Although the compositions are efficacious for treating many cancers, for some cancers, some subjects may have little or no response to the treatment. Therefore, remains a need for identifying subjects that could benefit from immunotherapy.

Accordingly, it is an object of the invention to provide compositions and methods for identifying subjects that will be responsive to or benefit from treatment with an immunotherapy that includes blockade of PD-1 signaling.

It is another object of the invention to provide methods of selecting a subject for the immunotherapy, or for selecting an alternative cancer therapy based on the subject's predicted responsiveness to the immunotherapy.

It is a further object of the invention to treat the subject with a suitable cancer treatment.

### SUMMARY OF THE INVENTION

Biomarkers predictive of a subject's likelihood of responding to an immunotherapy including blockade of PD-1 inhibitory signaling are disclosed. It has been discovered that expression of hypoxia-induced factor 1-alpha subunit (HIF1-Alpha) and kinase insert domain receptor (KDR) is significantly lower in cancer biopsies from subjects responsive to blockade of PD-1 inhibitory signaling compared to subjects that are non-responsive. Another two biomarkers, C-X-C motif chemokine 13 (CXCL13) and interleukin-7 receptor (IL7R) are expressed at significantly higher level in cancer biopsies from subjects responsive to blockade of PD-1 inhibitory signaling compared to subjects that are non-responsive.

Therefore, methods of identifying or selecting a subject who may benefit from treatment with an immunotherapy including blockade of PD-1 signaling and methods of predicting responsiveness of a subject suffering from cancer to treatment with an immunotherapy including blockade of PD-1 signaling are provided. The methods typically include determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a non-responder reference value, a responder reference value, or a combination thereof, wherein
(i) a level of HIF1-alpha in the sample obtained from the subject decreased compared to a non-responder reference value;

(ii) a level of HIF1-alpha in the sample obtained from the subject substantially the same or decreased compared a responder reference value;

(iii) a level of KDR in the sample obtained from the subject decreased compared to a non-responder reference value;
(iv) a level of KDR in the sample obtained from the subject substantially the same or decreased compared a responder reference value;

(v) a level of CXCL13 in the sample obtained from the subject increased compared to a non-responder reference value;

(vi) a level of CXCL13 in the sample obtained from the subject substantially the same or increased compared a responder reference value;

(vii) a level of IL7R in the sample obtained from the subject increased compared to a non-responder reference value;

(viii) a level of IL7R in the sample obtained from the subject substantially the same or increased compared a responder reference value; or

(ix) any combination thereof

indicates that the patient may benefit from treatment with the immunotherapy, or is likely to be responsive to the immunotherapy. Such subjects can be selected from treatment with the immunotherapy.

In some embodiments, two, three or all four of (i), (iii), (v), and (vii) are true. In some embodiments, one, two, three or all four of (ii), (iv), (vi), and (viii) are true.

Methods of determining the therapeutic efficacy of an immunotherapy including blockade of PD-1 signaling are also provided. The methods typically include determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a range of responsive references values to determine the therapeutic index of a particular immunotherapy for a particular cancer, wherein each of the responder reference values correlates with therapeutic efficacy on the immunotherapy.

Methods of determining that a subject is not likely to be responsive to, or not likely to benefit from an immunotherapy comprising blocking of PD-1 signaling are also disclosed. The methods typically include determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a non-responder reference value, a responder reference value, or a combination thereof, wherein
(i) a level of HIF1-alpha in the sample obtained from the subject substantial the same as or increased compared to a non-responder reference value;

(ii) a level of HIF1-alpha in the sample obtained from the subject increased compared a responder reference value;

(iii) a level of KDR in the sample obtained from the subject substantial the same as or increased compared to a non-responder reference value;

(iv) a level of KDR in the sample obtained from the subject increased compared a responder reference value;

(v) a level of CXCL13 in the sample obtained from the subject substantially the same or decreased compared to a non-responder reference value;
(vi) a level of CXCL13 in the sample obtained from the subject decreased compared a responder reference value;

(vii) a level of IL7R in the sample obtained from the subject substantially the same or decreased compared to a non-responder reference value;

(viii) a level of IL7R in the sample obtained from the subject decreased compared a responder reference value; or
(ix) any combination thereof

indicates the subject will not be responsive to, or will not exhibit benefit from the immunotherapy. Such subjects can be selected for a treatment other than the immunotherapy.

In some embodiments, two, three or all four of (ii), (iv), (vi), and (viii) are true. In some embodiments at least one, preferably two, three or all four of (i), (iii), (v), and (vii) are true.

Any of the disclosed methods can include determining the level of one, two, three or all four biomarkers, and comparing the level to a responder reference value, and non-responder reference value, or both.

Methods of detecting the levels of a biomarker in a sample are also provided. Typically, the expression level of a biomarker is determined by measuring the mRNA or protein level of the biomarker in the sample. Methods for measuring mRNA in a sample include, for example, quantitative polymerase chain reaction (qPCR), reverse transcription PCR (RT-PCR), reverse transcription real-time PCR (RT-qPCR), transcriptome analysis using next-generation sequencing, array hybridization analysis, digital PCR, Northern analysis, dot-blot, in situ hybridization, and RNase protection assay. Methods for measuring protein in a sample include, for example, immunoassay, ligand binding assay, mass spectroscopy, or high performance liquid chromatography (HPLC). Exemplary immunoassays include, but are not limited to, radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP).

Suitable biological samples and preparation thereof for analysis are also disclosed. Typically the biological sample includes one or more cancer cells. In preferred embodiments the sample is a tumor biopsy.

Methods of establishing reference values are also provided. For example, in some embodiments, a responder reference value is prepared by determining the protein or mRNA expression level of the biomarker(s) in a reference cancer cell sample obtained from a reference subject prior to treatment with the immunotherapy and wherein the immunotherapy was effective to slow cancer or tumor growth, prevent cancer or tumor growth, or reduce one or more symptoms of the cancer or tumor in the reference subject. Likewise, a non-responder reference value can be prepared by determining the protein or mRNA expression level of the biomarker(s) in a reference cancer cell sample obtained from a reference subject prior to treatment with the immunotherapy and wherein the immunotherapy was not effective to slow cancer or tumor growth, not effective to prevent cancer or tumor growth, or not effective to reduce one or more symptoms of the cancer or tumor in the reference subject.

Any of the methods can include a treatment step. For example, if the subject was determined to be responsive or to benefit from the immunotherapy, the treatment step can include administering the immunotherapy to the subject. If the subject was determined not to be responsive or not to benefit from the immunotherapy, the treatment step can include administering to the subject an alternative immunotherapy, or an alternative cancer therapy such as chemotherapy, radiation therapy, surgery, hormone therapy, photodynamic therapy, or anti-angiogenesis therapy.

Immunotherapies including blockade of PD-1 signaling are also provided. The immunotherapies typically include administering to the subject a PD-1 antagonist, such as an anti-PD-1 antibody, anti-B7-H1 antibody, or a B7-DC-Ig fusion protein, optionally a potentiating agent, and optionally a vaccine. Preferred embodiments include administering the subject a potentiating agent or a vaccine, and more preferred embodiments include administering the subject a potentiating agent and a vaccine. Preferred potentiating agents include, but are not limit to, a low dose of cyclophosphamide and anti-IL-10 antibody. Preferred vaccines include a tumor antigen, and optionally an adjuvant such as incomplete Freud's Adjuvant, GM-CSF, or a combination thereof.

### DETAILED DESCRIPTION OF THE INVENTION

### I. Definitions

As used herein, the term “biomarker” is anything that can be used as an indicator of a particular physiological state of an organism. For example a biomarker is the level(s) of a particular by-product, metabolite, mRNA or protein associated with the particular physiological state.

As used herein, the terms “optional” or “optionally” mean that the subsequently described event, circumstance, or material may or may not occur or be present, and that the description includes instances where the event, circumstance, or material occurs or is present and instances where it does not occur or is not present.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “treating” includes alleviating the symptoms associated with a specific disorder or condition and/or inhibiting the development or progression of the symptoms.

As used herein, the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the term “increase” can refer to a level including the reference level or cut-off-value or to an overall increase of 5%, 10%, 20%, 25%, 30%, 40%, 50%, 60%, 70%, 80%, 85%, 90%, 95%, 96%, 97%, 98%, 99% or greater, in biomarker level detected by the methods described herein, as compared to the level of the same biomarker from a reference sample. In certain embodiments, the term increase refers to the increase in biomarker level, wherein the increased level is 0.1, 0.5, 1, 2, 3, 4, 5-fold or more higher compared to the level of the biomarker in a reference sample.

As used herein, the term “decrease” can refer to a level below the reference level or cut-off-value or to an overall reduction of 5%, 10%, 20%, 25%, 30%, 40%, 50%, 60%, 70%, 80%, 85%, 90%, 95%, 96%, 97%, 98%, 99% or greater, in biomarker level detected by the methods described herein, as compared to the level of the same biomarker from a reference sample. In certain embodiments, the term decrease refers to the decrease in biomarker level, wherein the decreased level is 0.1, 0.5, 1, 2, 3, 4, 5-fold or more lower compared to the level of the biomarker in a reference sample.

At used herein, the term “at a reference level” refers to a biomarker level that is the same as the level of the same biomarker, detected by the methods described herein, from a reference sample.

As used herein, the term “reference level” herein refers to a predetermined value. As the skilled artisan will appreciate the reference level is predetermined and set to meet the requirements in terms of e.g. specificity and/or sensitivity. These requirements can vary, e.g. from regulatory body to regulatory body. It may, for example, be that assay sensitivity or specificity, respectively, has to be set to certain limits, e.g. 80%, 90% or 95%. These requirements may also be defined in terms of positive or negative predictive values. Nonetheless, based on the disclosure herein, it is possible to arrive at the reference level meeting those requirements.

As used herein, the phrases “substantially similar” or “substantially the same,” denote a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

As used herein, the phrases “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

As used herein, the term “inhibitory signal transduction” is intended to mean any signal transduction having the effect of abolishing, or otherwise reducing, T cell responses against an antigen, whether by reducing T cell proliferation or by any other inhibitory mechanism, whereby the extent or duration of an immunogenic T cell response is decreased. Such inhibitory signal transduction may be due to PD-1 binding to a natural ligand, such as binding of PD-1 by B7-H1 or some other member of this class of ligands, such as B7-DC.

The term “PD-1 antagonist” means any molecule that attenuates inhibitory signal transduction mediated by PD-1, found on the surface of T cells, B cells, natural killer (NK) cells, monocytes, DC, and macrophages. Such an antagonist includes a molecule that disrupts any inhibitory signal generated by a PD-1 molecule on a T cell. In specific examples, a PD-1 antagonist is a molecule that inhibits, reduces, abolishes or otherwise reduces inhibitory signal transduction through the PD-1 receptor signaling pathway. Such decrease may result where: (i) the PD-1 antagonist binds to a PD-1 receptor without triggering signal transduction, to reduce or block inhibitory signal transduction; (ii) the PD-1 antagonist binds to a ligand (e.g. an agonist) of the PD-1 receptor, preventing its binding thereto (for example, where said agonist is B7-H1); (iii) the PD-1 antagonist binds to, or otherwise inhibits the activity of, a molecule that is part of a regulatory chain that, when not inhibited, has the result of stimulating or otherwise facilitating PD-1 inhibitory signal transduction; or (iv) the PD-1 antagonist inhibits expression of a PD-1 receptor or expression ligand thereof, especially by reducing or abolishing expression of one or more genes encoding PD-1 or one or more of its natural ligands. Thus, a PD-1 antagonist is a molecule that effects a decrease in PD-1 inhibitory signal transduction, thereby increasing T cell response to one or more antigens.

As used herein, the term “active fragment” refers to a portion of a natural polypeptide, or a polypeptide with high sequence homology (for example, at least 80%, 85%, 90%, 95%, 98%, or 99% amino acid sequence identity) to a natural polypeptide and that exhibits PD-1 antagonist activity, for example, by binding PD-1 or by binding to a ligand of PD-1. In preferred embodiments, such a fragment would consist of the extracellular domain (ECD) of a B7-DC protein that binds to PD-1, preferably amino acids 20 to 221 thereof. In the case of PD-1 polypeptide, an active fragment would be a portion of said polypeptide comprising a binding domain that binds to a natural ligand of PD-1 to prevent stimulation of PD-1 mediated inhibitory signal transduction by said ligand. Active fragments may be identified by their ability to compete with the molecule they are derived from for binding to a natural binding site. For example, active fragments will compete with wild-type B7-DC for binding to PD-1.

With respect to an antibody, the term “active fragment” means an antigen binding portion of an antibody that is less than an entire immunoglobulin. Such fragments include Fab and F(ab₂)′ fragments, capable of reacting with and binding to any of the polypeptides disclosed herein as being receptors or ligands. These Fab and F(ab)₂ fragments lack the Fc portion of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

As used herein, the term “soluble portion” of a PD-1 antagonist means that portion of the full length polypeptide that does not include any part of the transmembrane portion or segment. For example, with respect to B7-DC, a soluble portion would include the extracellular portion (with or without the N-terminal signal sequence) but would not include any part of the transmembrane portion (or, at least, not enough to reduce solubility). Thus, the ECD of human B7-DC consists of both the IgV-like and IgC-like domains of the full length molecule (i.e., amino acids 20-221 of the full length sequence).

As used herein, a “co-stimulatory polypeptide” is a polypeptide that, upon interaction with a cell-surface molecule on T cells, modulates the activity of the T cell. Thus, the response of the T cell can be an effector (e.g., CTL or antibody-producing B cell) response, a helper response providing help for one or more effector (e.g., CTL or antibody-producing B cell) responses, or a suppressive response.

As used herein, the term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation). A polypeptide of the present invention may be a recombinant polypeptide, a natural polypeptide or a synthetic polypeptide, preferably a recombinant polypeptide.

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. A “fragment” of a polypeptide thus refers to any subset of the polypeptide that is a shorter polypeptide of the full length protein. Generally, fragments will be five or more amino acids in length.

As used herein, a derivative, analog or homolog, of a polypeptide (or fragment thereof) of the invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such derivatives and analogs are deemed to be within the scope of those skilled in the art from the teachings herein.

As used herein, the term “antibody” is meant to include both intact molecules as well as fragments thereof that include the antigen-binding site. Whole antibody structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al, J. Biol. Chem. 252:6609-6616 (1977). The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

### II. Methods for Selecting a Cancer Treatment

It has been discovered that genes are differentially regulated in cancer cells that are more responsive to immunotherapies including blockade of PD-1 signaling compared to cancer cells that are less responsive to immunotherapies including blockade of PD-1 signaling. Specifically, hypoxia-induced factor 1 alpha subunit (HIF1-alpha) and kinase insert domain receptor (KDR) have reduced expression and C-X-C motif chemokine 13 (CXCL13) and interleukin-7 receptor (IL7R) have increased expression in cancer cells that are more responsive to immunotherapies including blockade of PD-1 signaling compared to cancer cells that are less responsive to immunotherapies including blockade of PD-1 signaling. Accordingly, biomarkers for determining the efficacy of immunotherapies including blockade of PD-1 signaling are disclosed, and including HIF1-alpha, KDR, CXCL13, and IL7R.

As used herein an “immunotherapy including blockade of PD-1 signaling” refers to a therapy that includes administering a subject a compound that antagonizes PD-1 or a ligand thereof such as B7-H1. Exemplary antagonists include, but are not limited to, compounds that bind to and block PD-1 receptors on T cells without triggering inhibitory signal transduction, compounds that bind to PD-1 ligands to prevent their binding to PD-1, compounds that do both and compounds that prevent expression of genes that encode either PD-1 or natural ligands of PD-1. The blockade can be complete, or incomplete. Therefore, in some embodiments, “blockade of PD-1 signaling,” refers to inhibition of PD-1 signaling. In some embodiments, “blockade of PD-1 signaling” refers to reducing or decreasing PD-1 signaling. Immunotherapies that include blockade of PD-1 signaling are discussed in more detail below.

Methods of detecting the level of one or more biomarkers associated with a cancer cell's responsiveness to an immunotherapy including blockade of PD-1 signaling are disclosed. The methods can be used to identify cancer cells, and preferably tumors, in the subject that will be responsive to immunotherapies including blockade of PD-1 signaling. The methods can also be used to select subjects for treatment with an immunotherapy including blockade of PD-1 signaling, and to predict when an immunotherapy including blockade of PD-1 signaling is likely to be an effective treatment for a subject with cancer. The methods can also be used to aid in the selection of a treatment for a subject's cancer.

Kits and devices for use in the disclosed methods are also provided.

Compositions and methods of treating subjects with cancer are also provided. In some embodiments, the methods of treatment are coupled to a method of detection or selection.

A. Biomarkers for Determining the Efficacy of Immunotherapy

Biomarkers for determining the efficacy of immunotherapies including blockade of PD-1, and methods of use thereof are disclosed. As discussed in more detail below, in some embodiments, the methods include assaying one, two, three, or four of the biomarkers: hypoxia-induced factor 1-alpha subunit (HIF1-Alpha), kinase insert domain receptor (KDR), C-X-C motif chemokine 13 (CXCL13) and interleukin-7 receptor (IL7R).

1. Hypoxia-Induced Factor 1 Alpha Subunit (HIF1-Alpha)

Hypoxia-inducible factors (HIFs) are transcription factors that respond to a decrease in oxygen, or hypoxia, in the cellular environment. Oxygen-breathing species express the highly-conserved transcriptional complex HIF-1, which is a heterodimer composed of an alpha and a beta subunit, the latter being a constitutively-expressed aryl hydrocarbon receptor nuclear translocator (ARNT). HIF-1 belongs to the PER-ARNT-SIM (PAS) subfamily of the basic helix-loop-helix (bHLH) family of transcription factors. The alpha and beta subunit are similar in structure and both contain an N-terminal bHLH domain for DNA binding, a central region Per-ARNT-Sim (PAS) domain, which facilitates heterodimerization, and a C-terminal domain that recruits transcriptional coregulatory proteins.

Nucleic acid and amino acid sequence are known in the art. See, for example, NCBI Reference Sequence: NM_{—}001530.3, Homo sapiens hypoxia inducible factor 1, alpha subunit (basic helix-loop-helix transcription factor) (HIF1A), transcript variant 1, mRNA, which is specifically incorporated by reference in its entirety, and provides the HIF1-alpha mRNA sequence:

(SEQ ID NO: 1)
GCGCGCGCCGGCCTGGGCAGGCGAGCGGGCGCGCTCCCGCCCCCTCTCCC
CTCCCCGCGCGCCCGAGCGCGCCTCCGCCCTTGCCCGCCCCCTGACGCTG
CCTCAGCTCCTCAGTGCACAGTGCTGCCTCGTCTGAGGGGACAGGAGGAT
CACCCTCTTCGTCGCTTCGGCCAGTGTGTCGGGCTGGGCCCTGACAAGCC
ACCTGAGGAGAGGCTCGGAGCCGGGCCCGGACCCCGGCGATTGCCGCCCG
CTTCTCTCTAGTCTCACGAGGGGTTTCCCGCCTCGCACCCCCACCTCTGG
ACTTGCCTTTCCTTCTCTTCTCCGCGTGTGGAGGGAGCCAGCGCTTAGGC
CGGAGCGAGCCTGGGGGCCGCCCGCCGTGAAGACATCGCGGGGACCGATT
CACCATGGAGGGCGCCGGCGGCGCGAACGACAAGAAAAAGATAAGTTCTG
AACGTCGAAAAGAAAAGTCTCGAGATGCAGCCAGATCTCGGCGAAGTAAA
GAATCTGAAGTTTTTTATGAGCTTGCTCATCAGTTGCCACTTCCACATAA
TGTGAGTTCGCATCTTGATAAGGCCTCTGTGATGAGGCTTACCATCAGCT
ATTTGCGTGTGAGGAAACTTCTGGATGCTGGTGATTTGGATATTGAAGAT
GACATGAAAGCACAGATGAATTGCTTTTATTTGAAAGCCTTGGATGGTTT
TGTTATGGTTCTCACAGATGATGGTGACATGATTTACATTTCTGATAATG
TGAACAAATACATGGGATTAACTCAGTTTGAACTAACTGGACACAGTGTG
TTTGATTTTACTCATCCATGTGACCATGAGGAAATGAGAGAAATGCTTAC
ACACAGAAATGGCCTTGTGAAAAAGGGTAAAGAACAAAACACACAGCGAA
GCTTTTTTCTCAGAATGAAGTGTACCCTAACTAGCCGAGGAAGAACTATG
AACATAAAGTCTGCAACATGGAAGGTATTGCACTGCACAGGCCACATTCA
CGTATATGATACCAACAGTAACCAACCTCAGTGTGGGTATAAGAAACCAC
CTATGACCTGCTTGGTGCTGATTTGTGAACCCATTCCTCACCCATCAAAT
ATTGAAATTCCTTTAGATAGCAAGACTTTCCTCAGTCGACACAGCCTGGA
TATGAAATTTTCTTATTGTGATGAAAGAATTACCGAATTGATGGGATATG
AGCCAGAAGAACTTTTAGGCCGCTCAATTTATGAATATTATCATGCTTTG
GACTCTGATCATCTGACCAAAACTCATCATGATATGTTTACTAAAGGACA
AGTCACCACAGGACAGTACAGGATGCTTGCCAAAAGAGGTGGATATGTCT
GGGTTGAAACTCAAGCAACTGTCATATATAACACCAAGAATTCTCAACCA
CAGTGCATTGTATGTGTGAATTACGTTGTGAGTGGTATTATTCAGCACGA
CTTGATTTTCTCCCTTCAACAAACAGAATGTGTCCTTAAACCGGTTGAAT
CTTCAGATATGAAAATGACTCAGCTATTCACCAAAGTTGAATCAGAAGAT
ACAAGTAGCCTCTTTGACAAACTTAAGAAGGAACCTGATGCTTTAACTTT
GCTGGCCCCAGCCGCTGGAGACACAATCATATCTTTAGATTTTGGCAGCA
ACGACACAGAAACTGATGACCAGCAACTTGAGGAAGTACCATTATATAAT
GATGTAATGCTCCCCTCACCCAACGAAAAATTACAGAATATAAATTTGGC
AATGTCTCCATTACCCACCGCTGAAACGCCAAAGCCACTTCGAAGTAGTG
CTGACCCTGCACTCAATCAAGAAGTTGCATTAAAATTAGAACCAAATCCA
GAGTCACTGGAACTTTCTTTTACCATGCCCCAGATTCAGGATCAGACACC
TAGTCCTTCCGATGGAAGCACTAGACAAAGTTCACCTGAGCCTAATAGTC
CCAGTGAATATTGTTTTTATGTGGATAGTGATATGGTCAATGAATTCAAG
TTGGAATTGGTAGAAAAACTTTTTGCTGAAGACACAGAAGCAAAGAACCC
ATTTTCTACTCAGGACACAGATTTAGACTTGGAGATGTTAGCTCCCTATA
TCCCAATGGATGATGACTTCCAGTTACGTTCCTTCGATCAGTTGTCACCA
TTAGAAAGCAGTTCCGCAAGCCCTGAAAGCGCAAGTCCTCAAAGCACAGT
TACAGTATTCCAGCAGACTCAAATACAAGAACCTACTGCTAATGCCACCA
CTACCACTGCCACCACTGATGAATTAAAAACAGTGACAAAAGACCGTATG
GAAGACATTAAAATATTGATTGCATCTCCATCTCCTACCCACATACATAA
AGAAACTACTAGTGCCACATCATCACCATATAGAGATACTCAAAGTCGGA
CAGCCTCACCAAACAGAGCAGGAAAAGGAGTCATAGAACAGACAGAAAAA
TCTCATCCAAGAAGCCCTAACGTGTTATCTGTCGCTTTGAGTCAAAGAAC
TACAGTTCCTGAGGAAGAACTAAATCCAAAGATACTAGCTTTGCAGAATG
CTCAGAGAAAGCGAAAAATGGAACATGATGGTTCACTTTTTCAAGCAGTA
GGAATTGGAACATTATTACAGCAGCCAGACGATCATGCAGCTACTACATC
ACTTTCTTGGAAACGTGTAAAAGGATGCAAATCTAGTGAACAGAATGGAA
TGGAGCAAAAGACAATTATTTTAATACCCTCTGATTTAGCATGTAGACTG
CTGGGGCAATCAATGGATGAAAGTGGATTACCACAGCTGACCAGTTATGA
TTGTGAAGTTAATGCTCCTATACAAGGCAGCAGAAACCTACTGCAGGGTG
AAGAATTACTCAGAGCTTTGGATCAAGTTAACTGAGCTTTTTCTTAATTT
CATTCCTTTTTTTGGACACTGGTGGCTCATTACCTAAAGCAGTCTATTTA
TATTTTCTACATCTAATTTTAGAAGCCTGGCTACAATACTGCACAAACTT
GGTTAGTTCAATTTTGATCCCCTTTCTACTTAATTTACATTAATGCTCTT
TTTTAGTATGTTCTTTAATGCTGGATCACAGACAGCTCATTTTCTCAGTT
TTTTGGTATTTAAACCATTGCATTGCAGTAGCATCATTTTAAAAAATGCA
CCTTTTTATTTATTTATTTTTGGCTAGGGAGTTTATCCCTTTTTCGAATT
ATTTTTAAGAAGATGCCAATATAATTTTTGTAAGAAGGCAGTAACCTTTC
ATCATGATCATAGGCAGTTGAAAAATTTTTACACCTTTTTTTTCACATTT
TACATAAATAATAATGCTTTGCCAGCAGTACGTGGTAGCCACAATTGCAC
AATATATTTTCTTAAAAAATACCAGCAGTTACTCATGGAATATATTCTGC
GTTTATAAAACTAGTTTTTAAGAAGAAATTTTTTTTGGCCTATGAAATTG
TTAAACCTGGAACATGACATTGTTAATCATATAATAATGATTCTTAAATG
CTGTATGGTTTATTATTTAAATGGGTAAAGCCATTTACATAATATAGAAA
GATATGCATATATCTAGAAGGTATGTGGCATTTATTTGGATAAAATTCTC
AATTCAGAGAAATCATCTGATGTTTCTATAGTCACTTTGCCAGCTCAAAA
GAAAACAATACCCTATGTAGTTGTGGAAGTTTATGCTAATATTGTGTAAC
TGATATTAAACCTAAATGTTCTGCCTACCCTGTTGGTATAAAGATATTTT
GAGCAGACTGTAAACAAGAAAAAAAAAATCATGCATTCTTAGCAAAATTG
CCTAGTATGTTAATTTGCTCAAAATACAATGTTTGATTTTATGCACTTTG
TCGCTATTAACATCCTTTTTTTCATGTAGATTTCAATAATTGAGTAATTT
TAGAAGCATTATTTTAGGAATATATAGTTGTCACAGTAAATATCTTGTTT
TTTCTATGTACATTGTACAAATTTTTCATTCCTTTTGCTCTTTGTGGTTG
GATCTAACACTAACTGTATTGTTTTGTTACATCAAATAAACATCTTCTGT
GGACCAGGCAAAAAAAAAAAAAAAAAAAAAAA,

which includes an open reading frame that encodes a HIF1-alpha polypeptide having the sequence:

(SEQ ID NO: 2)
MEGAGGANDKKKISSERRKEKSRDAARSRRSKESEVFYELAHQLPLPHNV
SSHLDKASVMRLTISYLRVRKLLDAGDLDIEDDMKAQMNCFYLKALDGFV
MVLTDDGDMIYISDNVNKYMGLTQFELTGHSVFDFTHPCDHEEMREMLTH
RNGLVKKGKEQNTQRSFFLRMKCTLTSRGRTMNIKSATWKVLHCTGHIHV
YDTNSNQPQCGYKKPPMTCLVLICEPIPHPSNIEIPLDSKTFLSRHSLDM
KFSYCDERITELMGYEPEELLGRSIYEYYHALDSDHLTKTHHDMFTKGQV
TTGQYRMLAKRGGYVWVETQATVIYNTKNSQPQCIVCVNYVVSGIIQHDL
IFSLQQTECVLKPVESSDMKMTQLFTKVESEDTSSLFDKLKKEPDALTLL
APAAGDTIISLDFGSNDTETDDQQLEEVPLYNDVMLPSPNEKLQNINLAM
SPLPTAETPKPLRSSADPALNQEVALKLEPNPESLELSFTMPQIQDQTPS
PSDGSTRQSSPEPNSPSEYCFYVDSDMVNEFKLELVEKLFAEDTEAKNPF
STQDTDLDLEMLAPYIPMDDDFQLRSFDQLSPLESSSASPESASPQSTVT
VFQQTQIQEPTANATTTTATTDELKTVTKDRMEDIKILIASPSPTHIHKE
TTSATSSPYRDTQSRTASPNRAGKGVIEQTEKSHPRSPNVLSVALSQRTT
VPEEELNPKILALQNAQRKRKMEHDGSLFQAVGIGTLLQQPDDHAATTSL
SWKRVKGCKSSEQNGMEQKTTILTPSDLACRLLGQSMDESGLPQLTSYDC
EVNAPIQGSRNLLQGEELLRALDQVN.

Variants, including alternative splice variants, mutants, and other isoforms of SEQ ID NOS:1 and 2 are also known in the art. See, for example, UniProtKB accession number: Q16665 (HIF1A_HUMAN), which is specifically incorporated by reference in its entirety.

2. Kinase Insert Domain Receptor (KDR)

Kinase Insert Domain Receptor (KDR) also known as Vascular endothelial growth factor receptor 2 (VEGFR2) is a tyrosine-protein kinase that acts as a cell-surface receptor for VEGFA, VEGFC and VEGFD. KDR is believed to play an important role in the regulation of angiogenesis, vascular development, vascular permeability, and embryonic hematopoiesis, as well as promote proliferation, survival, migration and differentiation of endothelial cells. KDR is also thought to promote reorganization of the actin cytoskeleton. Isoforms lacking a transmembrane domain, such as isoform 2 and isoform 3, may function as decoy receptors for VEGFA, VEGFC and/or VEGFD.

Nucleic acid and amino acid sequence are known in the art. See, for example, NCBI Reference Sequence: NM_{—}002253.2, Homo sapiens kinase insert domain receptor (a type III receptor tyrosine kinase) (KDR), mRNA, which is specifically incorporated by reference in its entirety, and provides the KDR mRNA sequence:

(SEQ ID NO: 3)
ACTGAGTCCCGGGACCCCGGGAGAGCGGTCAATGTGTGGTCGCTGCGTTT
CCTCTGCCTGCGCCGGGCATCACTTGCGCGCCGCAGAAAGTCCGTCTGGC
AGCCTGGATATCCTCTCCTACCGGCACCCGCAGACGCCCCTGCAGCCGCG
GTCGGCGCCCGGGCTCCCTAGCCCTGTGCGCTCAACTGTCCTGCGCTGCG
GGGTGCCGCGAGTTCCACCTCCGCGCCTCCTTCTCTAGACAGGCGCTGGG
AGAAAGAACCGGCTCCCGAGTTCTGGGCATTTCGCCCGGCTCGAGGTGCA
GGATGCAGAGCAAGGTGCTGCTGGCCGTCGCCCTGTGGCTCTGCGTGGAG
ACCCGGGCCGCCTCTGTGGGTTTGCCTAGTGTTTCTCTTGATCTGCCCAG
GCTCAGCATACAAAAAGACATACTTACAATTAAGGCTAATACAACTCTTC
AAATTACTTGCAGGGGACAGAGGGACTTGGACTGGCTTTGGCCCAATAAT
CAGAGTGGCAGTGAGCAAAGGGTGGAGGTGACTGAGTGCAGCGATGGCCT
CTTCTGTAAGACACTCACAATTCCAAAAGTGATCGGAAATGACACTGGAG
CCTACAAGTGCTTCTACCGGGAAACTGACTTGGCCTCGGTCATTTATGTC
TATGTTCAAGATTACAGATCTCCATTTATTGCTTCTGTTAGTGACCAACA
TGGAGTCGTGTACATTACTGAGAACAAAAACAAAACTGTGGTGATTCCAT
GTCTCGGGTCCATTTCAAATCTCAACGTGTCACTTTGTGCAAGATACCCA
GAAAAGAGATTTGTTCCTGATGGTAACAGAATTTCCTGGGACAGCAAGAA
GGGCTTTACTATTCCCAGCTACATGATCAGCTATGCTGGCATGGTCTTCT
GTGAAGCAAAAATTAATGATGAAAGTTACCAGTCTATTATGTACATAGTT
GTCGTTGTAGGGTATAGGATTTATGATGTGGTTCTGAGTCCGTCTCATGG
AATTGAACTATCTGTTGGAGAAAAGCTTGTCTTAAATTGTACAGCAAGAA
CTGAACTAAATGTGGGGATTGACTTCAACTGGGAATACCCTTCTTCGAAG
CATCAGCATAAGAAACTTGTAAACCGAGACCTAAAAACCCAGTCTGGGAG
TGAGATGAAGAAATTTTTGAGCACCTTAACTATAGATGGTGTAACCCGGA
GTGACCAAGGATTGTACACCTGTGCAGCATCCAGTGGGCTGATGACCAAG
AAGAACAGCACATTTGTCAGGGTCCATGAAAAACCTTTTGTTGCTTTTGG
AAGTGGCATGGAATCTCTGGTGGAAGCCACGGTGGGGGAGCGTGTCAGAA
TCCCTGCGAAGTACCTTGGTTACCCACCCCCAGAAATAAAATGGTATAAA
AATGGAATACCCCTTGAGTCCAATCACACAATTAAAGCGGGGCATGTACT
GACGATTATGGAAGTGAGTGAAAGAGACACAGGAAATTACACTGTCATCC
TTACCAATCCCATTTCAAAGGAGAAGCAGAGCCATGTGGTCTCTCTGGTT
GTGTATGTCCCACCCCAGATTGGTGAGAAATCTCTAATCTCTCCTGTGGA
TTCCTACCAGTACGGCACCACTCAAACGCTGACATGTACGGTCTATGCCA
TTCCTCCCCCGCATCACATCCACTGGTATTGGCAGTTGGAGGAAGAGTGC
GCCAACGAGCCCAGCCAAGCTGTCTCAGTGACAAACCCATACCCTTGTGA
AGAATGGAGAAGTGTGGAGGACTTCCAGGGAGGAAATAAAATTGAAGTTA
ATAAAAATCAATTTGCTCTAATTGAAGGAAAAAACAAAACTGTAAGTACC
CTTGTTATCCAAGCGGCAAATGTGTCAGCTTTGTACAAATGTGAAGCGGT
CAACAAAGTCGGGAGAGGAGAGAGGGTGATCTCCTTCCACGTGACCAGGG
GTCCTGAAATTACTTTGCAACCTGACATGCAGCCCACTGAGCAGGAGAGC
GTGTCTTTGTGGTGCACTGCAGACAGATCTACGTTTGAGAACCTCACATG
GTACAAGCTTGGCCCACAGCCTCTGCCAATCCATGTGGGAGAGTTGCCCA
CACCTGTTTGCAAGAACTTGGATACTCTTTGGAAATTGAATGCCACCATG
TTCTCTAATAGCACAAATGACATTTTGATCATGGAGCTTAAGAATGCATC
CTTGCAGGACCAAGGAGACTATGTCTGCCTTGCTCAAGACAGGAAGACCA
AGAAAAGACATTGCGTGGTCAGGCAGCTCACAGTCCTAGAGCGTGTGGCA
CCCACGATCACAGGAAACCTGGAGAATCAGACGACAAGTATTGGGGAAAG
CATCGAAGTCTCATGCACGGCATCTGGGAATCCCCCTCCACAGATCATGT
GGTTTAAAGATAATGAGACCCTTGTAGAAGACTCAGGCATTGTATTGAAG
GATGGGAACCGGAACCTCACTATCCGCAGAGTGAGGAAGGAGGACGAAGG
CCTCTACACCTGCCAGGCATGCAGTGTTCTTGGCTGTGCAAAAGTGGAGG
CATTTTTCATAATAGAAGGTGCCCAGGAAAAGACGAACTTGGAAATCATT
ATTCTAGTAGGCACGGCGGTGATTGCCATGTTCTTCTGGCTACTTCTTGT
CATCATCCTACGGACCGTTAAGCGGGCCAATGGAGGGGAACTGAAGACAG
GCTACTTGTCCATCGTCATGGATCCAGATGAACTCCCATTGGATGAACAT
TGTGAACGACTGCCTTATGATGCCAGCAAATGGGAATTCCCCAGAGACCG
GCTGAAGCTAGGTAAGCCTCTTGGCCGTGGTGCCTTTGGCCAAGTGATTG
AAGCAGATGCCTTTGGAATTGACAAGACAGCAACTTGCAGGACAGTAGCA
GTCAAAATGTTGAAAGAAGGAGCAACACACAGTGAGCATCGAGCTCTCAT
GTCTGAACTCAAGATCCTCATTCATATTGGTCACCATCTCAATGTGGTCA
ACCTTCTAGGTGCCTGTACCAAGCCAGGAGGGCCACTCATGGTGATTGTG
GAATTCTGCAAATTTGGAAACCTGTCCACTTACCTGAGGAGCAAGAGAAA
TGAATTTGTCCCCTACAAGACCAAAGGGGCACGATTCCGTCAAGGGAAAG
ACTACGTTGGAGCAATCCCTGTGGATCTGAAACGGCGCTTGGACAGCATC
ACCAGTAGCCAGAGCTCAGCCAGCTCTGGATTTGTGGAGGAGAAGTCCCT
CAGTGATGTAGAAGAAGAGGAAGCTCCTGAAGATCTGTATAAGGACTTCC
TGACCTTGGAGCATCTCATCTGTTACAGCTTCCAAGTGGCTAAGGGCATG
GAGTTCTTGGCATCGCGAAAGTGTATCCACAGGGACCTGGCGGCACGAAA
TATCCTCTTATCGGAGAAGAACGTGGTTAAAATCTGTGACTTTGGCTTGG
CCCGGGATATTTATAAAGATCCAGATTATGTCAGAAAAGGAGATGCTCGC
CTCCCTTTGAAATGGATGGCCCCAGAAACAATTTTTGACAGAGTGTACAC
AATCCAGAGTGACGTCTGGTCTTTTGGTGTTTTGCTGTGGGAAATATTTT
CCTTAGGTGCTTCTCCATATCCTGGGGTAAAGATTGATGAAGAATTTTGT
AGGCGATTGAAAGAAGGAACTAGAATGAGGGCCCCTGATTATACTACACC
AGAAATGTACCAGACCATGCTGGACTGCTGGCACGGGGAGCCCAGTCAGA
GACCCACGTTTTCAGAGTTGGTGGAACATTTGGGAAATCTCTTGCAAGCT
AATGCTCAGCAGGATGGCAAAGACTACATTGTTCTTCCGATATCAGAGAC
TTTGAGCATGGAAGAGGATTCTGGACTCTCTCTGCCTACCTCACCTGTTT
CCTGTATGGAGGAGGAGGAAGTATGTGACCCCAAATTCCATTATGACAAC
ACAGCAGGAATCAGTCAGTATCTGCAGAACAGTAAGCGAAAGAGCCGGCC
TGTGAGTGTAAAAACATTTGAAGATATCCCGTTAGAAGAACCAGAAGTAA
AAGTAATCCCAGATGACAACCAGACGGACAGTGGTATGGTTCTTGCCTCA
GAAGAGCTGAAAACTTTGGAAGACAGAACCAAATTATCTCCATCTTTTGG
TGGAATGGTGCCCAGCAAAAGCAGGGAGTCTGTGGCATCTGAAGGCTCAA
ACCAGACAAGCGGCTACCAGTCCGGATATCACTCCGATGACACAGACACC
ACCGTGTACTCCAGTGAGGAAGCAGAACTTTTAAAGCTGATAGAGATTGG
AGTGCAAACCGGTAGCACAGCCCAGATTCTCCAGCCTGACTCGGGGACCA
CACTGAGCTCTCCTCCTGTTTAAAAGGAAGCATCCACACCCCCAACTCCT
GGACATCACATGAGAGGTGCTGCTCAGATTTTCAAGTGTTGTTCTTTCCA
CCAGCAGGAAGTAGCCGCATTTGATTTTCATTTCGACAACAGAAAAAGGA
CCTCGGACTGCAGGGAGCCAGTCTTCTAGGCATATCCTGGAAGAGGCTTG
TGACCCAAGAATGTGTCTGTGTCTTCTCCCAGTGTTGACCTGATCCTCTT
TTTCATTCATTTAAAAAGCATTTATCATGCCCCCTGCTGCGGGTCTCACC
ATGGGTTTAGAACAAAGACGTTCAAGAAATGGCCCCATCCTCAAAGAAGT
AGCAGTACCTGGGGAGCTGACACTTCTGTAAAACTAGAAGATAAACCAGG
CAATGTAAGTGTTCGAGGTGTTGAAGATGGGAAGGATTTGCAGGGCTGAG
TCTATCCAAGAGGCTTTGTTTAGGACGTGGGTCCCAAGCCAAGCCTTAAG
TGTGGAATTCGGATTGATAGAAAGGAAGACTAACGTTACCTTGCTTTGGA
GAGTACTGGAGCCTGCAAATGCATTGTGTTTGCTCTGGTGGAGGTGGGCA
TGGGGTCTGTTCTGAAATGTAAAGGGTTCAGACGGGGTTTCTGGTTTTAG
AAGGTTGCGTGTTCTTCGAGTTGGGCTAAAGTAGAGTTCGTTGTGCTGTT
TCTGACTCCTAATGAGAGTTCCTTCCAGACCGTTACGTGTCTCCTGGCCA
AGCCCCAGGAAGGAAATGATGCAGCTCTGGCTCCTTGTCTCCCAGGCTGA
TCCTTTATTCAGAATACCACAAAGAAAGGACATTCAGCTCAAGGCTCCCT
GCCGTGTTGAAGAGTTCTGACTGCACAAACCAGCTTCTGGTTTCTTCTGG
AATGAATACCCTCATATCTGTCCTGATGTGATATGTCTGAGACTGAATGC
GGGAGGTTCAATGTGAAGCTGTGTGTGGTGTCAAAGTTTCAGGAAGGATT
TTACCCTTTTGTTCTTCCCCCTGTCCCCAACCCACTCTCACCCCGCAACC
CATCAGTATTTTAGTTATTTGGCCTCTACTCCAGTAAACCTGATTGGGTT
TGTTCACTCTCTGAATGATTATTAGCCAGACTTCAAAATTATTTTATAGC
CCAAATTATAACATCTATTGTATTATTTAGACTTTTAACATATAGAGCTA
TTTCTACTGATTTTTGCCCTTGTTCTGTCCTTTTTTTCAAAAAAGAAAAT
GTGTTTTTTGTTTGGTACCATAGTGTGAAATGCTGGGAACAATGACTATA
AGACATGCTATGGCACATATATTTATAGTCTGTTTATGTAGAAACAAATG
TAATATATTAAAGCCTTATATATAATGAACTTTGTACTATTCACATTTTG
TATCAGTATTATGTAGCATAACAAAGGTCATAATGCTTTCAGCAATTGAT
GTCATTTTATTAAAGAACATTGAAAAACTTGAAGGAATCCCTTTGCAAGG
TTGCATTACTGTACCCATCATTTCTAAAATGGAAGAGGGGGTGGCTGGGC
ACAGTGGCCGACACCTAAAAACCCAGCACTTTGGGGGGCCAAGGTGGGAG
GATCGCTTGAGCCCAGGAGTTCAAGACCAGTCTGGCCAACATGGTCAGAT
TCCATCTCAAAGAAAAAAGGTAAAAATAAAATAAAATGGAGAAGAAGGAA
TCAGA,

(SEQ ID NO:3), which includes an open reading frame that encodes a KDR polypeptide having the amino acid sequence:

(SEQ ID NO: 4)
MQSKVLLAVALWLCVETRAASVGLPSVSLDLPRLSIQKDILTIKANTTLQ
ITCRGQRDLDWLWPNNQSGSEQRVEVTECSDGLFCKTLTIPKVIGNDTGA
YKCFYRETDLASVIYVYVQDYRSPFIASVSDQHGVVYITENKNKTVVIPC
LGSISNLNVSLCARYPEKRFVPDGNRISWDSKKGFTIPSYMISYAGMVFC
EAKINDESYQSIMYIVVVVGYRIYDVVLSPSHGIELSVGEKLVLNCTART
ELNVGIDFNWEYPSSKHQHKKLVNRDLKTQSGSEMKKFLSTLTIDGVTRS
DQGLYTCAASSGLMTKKNSTFVRVHEKPFVAFGSGMESLVEATVGERVRI
PAKYLGYPPPEIKWYKNGIPLESNHTIKAGHVLTIMEVSERDTGNYTVIL
TNPISKEKQSHVVSLVVYVPPQIGEKSLISPVDSYQYGTTQTLTCTVYAI
PPPHHIHWYWQLEEECANEPSQAVSVTNPYPCEEWRSVEDFQGGNKIEVN
KNQFALIEGKNKTVSTLVIQAANVSALYKCEAVNKVGRGERVISFHVTRG
PEITLQPDMQPTEQESVSLWCTADRSTFENLTWYKLGPQPLPIHVGELPT
PVCKNLDTLWKLNATMFSNSTNDILIMELKNASLQDQGDYVCLAQDRKTK
KRHCVVRQLTVLERVAPTITGNLENQTTSIGESIEVSCTASGNPPPQIMW
FKDNETLVEDSGIVLKDGNRNLTIRRVRKEDEGLYTCQACSVLGCAKVEA
FFIIEGAQEKTNLEIIILVGTAVIAMFFWLLLVIILRTVKRANGGELKTG
YLSIVMDPDELPLDEHCERLPYDASKWEFPRDRLKLGKPLGRGAFGQVIE
ADAFGIDKTATCRTVAVKMLKEGATHSEHRALMSELKILIHIGHHLNVVN
LLGACTKPGGPLMVIVEFCKFGNLSTYLRSKRNEFVPYKTKGARFRQGKD
YVGAIPVDLKRRLDSITSSQSSASSGFVEEKSLSDVEEEEAPEDLYKDFL
TLEHLICYSFQVAKGMEFLASRKCIHRDLAARNILLSEKNVVKICDFGLA
RDIYKDPDYVRKGDARLPLKWMAPETIFDRVYTIQSDVWSFGVLLWEIFS
LGASPYPGVKIDEEFCRRLKEGTRMRAPDYTTPEMYQTMLDCWHGEPSQR
PTFSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPEVK
VIPDDNQTDSGMVLASEELKTLEDRTKLSPSFGGMVPSKSRESVASEGSN
QTSGYQSGYHSDDTDTTVYSSEEAELLKLIEIGVQTGSTAQILQPDSGTT
LSSPPV.

Variants, including alternative splice variants, mutants, and other isoforms of SEQ ID NOS:3 and 4 are also known in the art. See, for example, UniProtKB accession number: P35968 (VGFR2_HUMAN), which is specifically incorporated by reference in its entirety.

3. C-X-C Motif Chemokine 13 (CXCL13)

C-X-C motif chemokine 13 (CXCL13) is a small cytokine belonging to the CXC chemokine family. It is selectively chemotactic for B cells belonging to both the B-1 and B-2 subsets, and elicits its effects by interacting with chemokine receptor CXCR5. CXCL13 and its receptor CXCR5 control the organization of B cells within follicles of lymphoid tissues.

Nucleic acid and amino acid sequences for CXCL13 are known in the art. See, for example, NCBI Reference Sequence: NM_{—}006419.2, Homo sapiens chemokine (C-X-C motif) ligand 13 (CXCL13), mRNA which is specifically incorporated by reference in its entirety, and provides the CXCL13 mRNA sequence:

(SEQ ID NO: 5)
GAGAAGATGTTTGAAAAAACTGACTCTGCTAATGAGCCTGGACTCAGAGC
TCAAGTCTGAACTCTACCTCCAGACAGAATGAAGTTCATCTCGACATCTC
TGCTTCTCATGCTGCTGGTCAGCAGCCTCTCTCCAGTCCAAGGTGTTCTG
GAGGTCTATTACACAAGCTTGAGGTGTAGATGTGTCCAAGAGAGCTCAGT
CTTTATCCCTAGACGCTTCATTGATCGAATTCAAATCTTGCCCCGTGGGA
ATGGTTGTCCAAGAAAAGAAATCATAGTCTGGAAGAAGAACAAGTCAATT
GTGTGTGTGGACCCTCAAGCTGAATGGATACAAAGAATGATGGAAGTATT
GAGAAAAAGAAGTTCTTCAACTCTACCAGTTCCAGTGTTTAAGAGAAAGA
TTCCCTGATGCTGATATTTCCACTAAGAACACCTGCATTCTTCCCTTATC
CCTGCTCTGGATTTTAGTTTTGTGCTTAGTTAAATCTTTTCCAGGAAAAA
GAACTTCCCCATACAAATAAGCATGAGACTATGTAAAAATAACCTTGCAG
AAGCTGATGGGGCAAACTCAAGCTTCTTCACTCACAGCACCCTATATACA
CTTGGAGTTTGCATTCTTATTCATCAGGGAGGAAAGTTTCTTTGAAAATA
GTTATTCAGTTATAAGTAATACAGGATTATTTTGATTATATACTTGTTGT
TTAATGTTTAAAATTTCTTAGAAAACAATGGAATGAGAATTTAAGCCTCA
AATTTGAACATGTGGCTTGAATTAAGAAGAAAATTATGGCATATATTAAA
AGCAGGCTTCTATGAAAGACTCAAAAAGCTGCCTGGGAGGCAGATGGAAC
TTGAGCCTGTCAAGAGGCAAAGGAATCCATGTAGTAGATATCCTCTGCTT
AAAAACTCACTACGGAGGAGAATTAAGTCCTACTTTTAAAGAATTTCTTT
ATAAAATTTACTGTCTAAGATTAATAGCATTCGAAGATCCCCAGACTTCA
TAGAATACTCAGGGAAAGCATTTAAAGGGTGATGTACACATGTATCCTTT
CACACATTTGCCTTGACAAACTTCTTTCACTCACATCTTTTTCACTGACT
TTTTTTGTGGGGGGCGGGGCCGGGGGGACTCTGGTATCTAATTCTTTAAT
GATTCCTATAAATCTAATGACATTCAATAAAGTTGAGCAAACATTTTACT
TAAAAAAAAAAAAAAAAAA,

which includes an open reading frame that encodes a CXCL13 polypeptide having the amino acid sequence:

(SEQ ID NO: 6)
MKFISTSLLLMLLVSSLSPVQGVLEVYYTSLRCRCVQESSVFIPRRFIDR
IQILPRGNGCPRKEIIVWKKNKSIVCVDPQAEWIQRMMEVLRKRSSSTLP
VPVFKRKIP.

See also UniProtKB accession number: 043927 (CXL13_HUMAN), which is specifically incorporated by reference in its entirety.

4. Interleukin-7 Receptor (IL7R)

The interleukin-7 receptor is a cell surface heterodimer consisting of two subunits, interleukin-7 receptor-α (CD127) and common-γ chain receptor (CD132). The common-γ chain receptors is shared with various cytokines, including interleukin-2, -4, -9, and -15. Interleukin-7 receptor is expressed on various cell types, including naive and memory T cells and many others. Interleukin-7 receptor has been shown to play an important function in the development of lymphocytes by regulating V(D)J recombination. The protein is also found to control the accessibility of a region of the genome that contains the T-cell receptor gamma gene, by STATS and histone acetylation.

Nucleic acid and amino acid sequences for interleukin-7 receptor-α (CD127) are known in the art. See, for example, NCBI Reference Sequence: NM_{—}002185.3, Homo sapiens interleukin 7 receptor (IL7R), mRNA, which is specifically incorporated by reference in its entirety, and provides the interleukin-7 receptor-α (CD127) mRNA sequence:

(SEQ ID NO: 7)
ATCTAAGCTTCTCTGTCTTCCTCCCTCCCTCCCTTCCTCTTACTCTCATT
CATTTCATACACACTGGCTCACACATCTACTCTCTCTCTCTATCTCTCTC
AGAATGACAATTCTAGGTACAACTTTTGGCATGGTTTTTTCTTTACTTCA
AGTCGTTTCTGGAGAAAGTGGCTATGCTCAAAATGGAGACTTGGAAGATG
CAGAACTGGATGACTACTCATTCTCATGCTATAGCCAGTTGGAAGTGAAT
GGATCGCAGCACTCACTGACCTGTGCTTTTGAGGACCCAGATGTCAACAT
CACCAATCTGGAATTTGAAATATGTGGGGCCCTCGTGGAGGTAAAGTGCC
TGAATTTCAGGAAACTACAAGAGATATATTTCATCGAGACAAAGAAATTC
TTACTGATTGGAAAGAGCAATATATGTGTGAAGGTTGGAGAAAAGAGTCT
AACCTGCAAAAAAATAGACCTAACCACTATAGTTAAACCTGAGGCTCCTT
TTGACCTGAGTGTCGTCTATCGGGAAGGAGCCAATGACTTTGTGGTGACA
TTTAATACATCACACTTGCAAAAGAAGTATGTAAAAGTTTTAATGCACGA
TGTAGCTTACCGCCAGGAAAAGGATGAAAACAAATGGACGCATGTGAATT
TATCCAGCACAAAGCTGACACTCCTGCAGAGAAAGCTCCAACCGGCAGCA
ATGTATGAGATTAAAGTTCGATCCATCCCTGATCACTATTTTAAAGGCTT
CTGGAGTGAATGGAGTCCAAGTTATTACTTCAGAACTCCAGAGATCAATA
ATAGCTCAGGGGAGATGGATCCTATCTTACTAACCATCAGCATTTTGAGT
TTTTTCTCTGTCGCTCTGTTGGTCATCTTGGCCTGTGTGTTATGGAAAAA
AAGGATTAAGCCTATCGTATGGCCCAGTCTCCCCGATCATAAGAAGACTC
TGGAACATCTTTGTAAGAAACCAAGAAAAAATTTAAATGTGAGTTTCAAT
CCTGAAAGTTTCCTGGACTGCCAGATTCATAGGGTGGATGACATTCAAGC
TAGAGATGAAGTGGAAGGTTTTCTGCAAGATACGTTTCCTCAGCAACTAG
AAGAATCTGAGAAGCAGAGGCTTGGAGGGGATGTGCAGAGCCCCAACTGC
CCATCTGAGGATGTAGTCATCACTCCAGAAAGCTTTGGAAGAGATTCATC
CCTCACATGCCTGGCTGGGAATGTCAGTGCATGTGACGCCCCTATTCTCT
CCTCTTCCAGGTCCCTAGACTGCAGGGAGAGTGGCAAGAATGGGCCTCAT
GTGTACCAGGACCTCCTGCTTAGCCTTGGGACTACAAACAGCACGCTGCC
CCCTCCATTTTCTCTCCAATCTGGAATCCTGACATTGAACCCAGTTGCTC
AGGGTCAGCCCATTCTTACTTCCCTGGGATCAAATCAAGAAGAAGCATAT
GTCACCATGTCCAGCTTCTACCAAAACCAGTGAAGTGTAAGAAACCCAGA
CTGAACTTACCGTGAGCGACAAAGATGATTTAAAAGGGAAGTCTAGAGTT
CCTAGTCTCCCTCACAGCACAGAGAAGACAAAATTAGCAAAACCCCACTA
CACAGTCTGCAAGATTCTGAAACATTGCTTTGACCACTCTTCCTGAGTTC
AGTGGCACTCAACATGAGTCAAGAGCATCCTGCTTCTACCATGTGGATTT
GGTCACAAGGTTTAAGGTGACCCAATGATTCAGCTATTTAAAAAAAAAAG
AGGAAAGAATGAAAGAGTAAAGGAAATGATTGAGGAGTGAGGAAGGCAGG
AAGAGAGCATGAGAGGAAAGAAAGAAAGGAAAATAAAAAATGATAGTTGC
CATTATTAGGATTTAATATATATCCAGTGCTTTGCAAGTGCTCTGCGCAC
CTTGTCTCACTCCATCCTGACAATAATCCTGGGAGGTGTGTGCAATTACT
ACGACTACTCTCTTTTTTATAGATCATTAAATTCAGAACTAAGGAGTTAA
GTAACTTGTCCAAGTTGTTCACACAGTGAAGGGAGGGGCCAAGATATGAT
GGCTGGGAGTCTAATTGCAGTTCCCTGAGCCATGTGCCTTTCTCTTCACT
GAGGACTGCCCCATTCTTGAGTGCCAAACGTCACTAGTAACAGGGTGTGC
CTAGATAATTTATGATCCAAACTGAGTCAGTTTGGAAAGTGAAAGGGAAA
CTTACATATAATCCCTCCGGGACAATGAGCAAAAACTAGGACTGTCCCCA
GACAAATGTGAACATACATATCATCACTTAAATTAAAATGGCTATGAGAA
AGAAAGAGGGGGAGAAACAGTCTTGCGGGTGTGAAGTCCCATGACCAGCC
ATGTCAAAAGAAGGTAAAGAAGTCAAGAAAAAGCCATGAAGCCCATTTGG
TTTCATTTTTCTGAAAATAGGCTCAAGAGGGAATAAATTAGAAACTCACA
ATTTCTCTTGTTTGTTACCAAGACAGTGATTCTCTTGCTGCTACCACCCA
ACTGCATCCGTCCATGATCTCAGAGGAAACTGTCGCTGACCCTGGACATG
GGTACGTTTGACGAGTGAGAGGAGGCATGACCCCTCCCATGTGTATAGAC
ACTACCCCAACCTAAATTCATCCCTAAATTGTCCCAAGTTCTCCAGCAAT
AGAGGCTGCCACAAACTTCAGGGAGAAAGAGTTACAAGTACATGCAATGA
GTGAACTGACTGTGGCTACAATCTTGAAGATATACGGAAGAGACGTATTA
TTAATGCTTGACATATATCATCTTGCCTTTCTTGGTCTAGACTGACTTCT
AATGACTAACTCAAAGTCAAGGCAACTGAGTAATGTCAGCTCAGCAAAGT
GCAGCAAACCCATCTCCCACAGGCCTCCAAACCCTGGCTGTTCACAGAAC
CACAAAGGGCAGATGCTGCACAGAAAACTAGAGAAGGGGTCATAGGTTCA
TGGTTTTGTTTGAGATTTGTTGCTACTGTTTTTCTGTTTTGAATTTTCTT
CTTTGTTCTGTTTTTACTTTATTTAGGGGGACTAGGTGTTTCTGATATTT
TAGTTTTCTTGTTTGTTTTGTTTTGTGTTGTCTGTGAATGGGGTTTTAAC
TGTGGATGAATGGACCTTATCTGTTGGCTTAAAGGACTGGTAAGATCAGA
CCATCTTATTCTTCAGGTGAATGTTTTACTTTCCAAAGTGCTCTCCTCTG
CACCAGCAGTAATAAATACAATGCCATAATCCCTTAGGTTTGCCTAGTGC
TTTTGCAATTTTCAAAGCACTTCCATAAGCATTCCTTCCACCTCCTTGAT
AGGCATTTATGGAAAGCCTGCTACATGTCAATCATACTGTTAGGCACAGG
GGACCTAAAGACACATAAAAGGATGGCATTCTGCCTCATAAATTGCAAAA
CCTAATGAAAGTGACTGCTTGGTAAACAAATTATTATTATATTATAAAAT
GCTATAAAAGAGCCATATTGAAAGTGCCCTGTTGGAGACAGGGCAAATGC
CACAAAAATGATGTAAATTTACATGGAGGAAAAGTAGAATCTGCCTGGTT
TGTAGGCAGCAGAAGACATTTTTCATCAGTGGGCAGGTGTTCTTTACCTT
TTGTAGAAATGGGAGTCAAGTCTCAAATAGGAGGCTCCACAAAATCTCAT
GCCAGGTCTCTGATACCTTATTCACAGAAGTTCTTTGAAGTATTTATTGT
TATTTTCTTTGACTTATGGGAAAACTGGGACACAGGAAGACAGGTAAATT
ACCCAACCTCACACGTTAAGTCAGAACTGGGAGCCATAATTTTGTATCCC
TGGTATAAATAGACAATCTCTTGAAGAAATGAAGAGATGACCATAGAAAA
ACATCGAGATATCTCCAGCTCTAAAATCCTTTGTTTCAATGTTGTTTGGC
ATATGTTATCTTTGGAATTTAGTGTCTGAGCCTCTGTCTGTTACTGTAGT
ATTTAAAATGCATGTATTATAATCATATAATCATAACTGCTGTTAATTCT
TGATTATATACCTAGGGACAATGTGTAATGTAAGATTACTAATTGGTTCT
GCCCAATCTCCTTTCAGATTTTATTAGGAAAAAAAAATAAACCTCCTGAT
CGGAGACAATGTATTAATCAGAAGTGTAAACTGCCAGTTCTATATAGCAT
GAAATGAAAAGACAGCTAATTTGGTCCAACAAACATGACTGGGTCTAGGG
CACCCAGGCTGATTCAGCTGATTTCCTACCAGCCTTTGCCTCTTCCTTCA
ATGTGGTTTCCATGGGAATTTGCTTCAGAAAAGCCAAGTATGGGCTGTTC
AGAGGTGCACACCTGCATTTTCTTAGCTCTTCTAGAGGGGCTAAGAGACT
TGGTACGGGCCAGGAAGAATATGTGGCAGAGCTCCTGGAAATGATGCAGA
TTAGGTGGCATTTTTGTCAGCTCTGTGGTTTATTGTTGGGACTATTCTTT
AAAATATCCATTGTTCACTACAGTGAAGATCTCTGATTTAACCGTGTACT
ATCCACATGCATTACAAACATTTCGCAGAGCTGCTTAGTATATAAGCGTA
CAATGTATGTAATAACCATCTCATATTTAATTAAATGGTATAGAAGAACA
AAAAAAAAAAAAAAAAA,

which includes an open reading frame that encodes a interleukin-7 receptor-α (CD127) polypeptide having the amino acid sequence:

(SEQ ID NO: 8)
MTILGTTFGMVFSLLQVVSGESGYAQNGDLEDAELDDYSFSCYSQLEVNG
SQHSLTCAFEDPDVNITNLEFEICGALVEVKCLNFRKLQEIYFIETKKFL
LIGKSNICVKVGEKSLTCKKIDLTTIVKPEAPFDLSVVYREGANDFVVTF
NTSHLQKKYVKVLMHDVAYRQEKDENKWTHVNLSSTKLTLLQRKLQPAAM
YEIKVRSIPDHYFKGFWSEWSPSYYFRTPEINNSSGEMDPILLTISILSF
FSVALLVILACVLWKKRIKPIVWPSLPDHKKTLEHLCKKPRKNLNVSFNP
ESFLDCQIHRVDDIQARDEVEGFLQDTFPQQLEESEKQRLGGDVQSPNCP
SEDVVITPESFGRDSSLTCLAGNVSACDAPILSSSRSLDCRESGKNGPHV
YQDLLLSLGTTNSTLPPPFSLQSGILTLNPVAQGQPILTSLGSNQEEAYV
TMSSFYQNQ.

A consensus amino acid sequence for interleukin-7 receptor-α (CD127) is

(SEQ ID NO: 9)
MTILGTTFGMVFSLLQVVSGESGYAQNGDLEDAELDDYSFSCYSQLEVNG
SQHSLTCAFEDPDVNTTNLEFEICGALVEVKCLNFRKLQEIYFIETKKFL
LIGKSNICVKVGEKSLTCKKIDLTTIVKPEAPFDLSVIYREGANDFVVTF
NTSHLQKKYVKVLMHDVAYRQEKDENKWTHVNLSSTKLTLLQRKLQPAAM
YEIKVRSIPDHYFKGFWSEWSPSYYFRTPEINNSSGEMDPILLTISILSF
FSVALLVILACVLWKKRIKPIVWPSLPDHKKTLEHLCKKPRKNLNVSFNP
ESFLDCQIHRVDDIQARDEVEGFLQDTFPQQLEESEKQRLGGDVQSPNCP
SEDVVITPESFGRDSSLTCLAGNVSACDAPILSSSRSLDCRESGKNGPHV
YQDLLLSLGTTNSTLPPPFSLQSGILTLNPVAQGQPILTSLGSNQEEAYV
TMSSFYQNQ,

which is a variant of SEQ ID NO:8.

Variants, including alternative splice variants, mutants, and alternative isoforms of SEQ ID NOS:7, 8 and 9 are also known in the art. See, for example, UniProtKB accession number: P16871 (IL7RA_HUMAN), which is specifically incorporated by reference in its entirety.

Nucleic acid and amino acid sequences for common-γ chain receptor (CD132) are known in the art. See, for example, NCBI Reference Sequence: NM_{—}000206.2, Homo sapiens interleukin 2 receptor, gamma (IL2RG), mRNA, which is specifically incorporated by reference in its entirety, and provides the common-γ chain receptor (CD132) mRNA sequence:

(SEQ ID NO: 10)
AGAGGAAACGTGTGGGTGGGGAGGGGTAGTGGGTGAGGGACCCAGGTTCC
TGACACAGACAGACTACACCCAGGGAATGAAGAGCAAGCGCCATGTTGAA
GCCATCATTACCATTCACATCCCTCTTATTCCTGCAGCTGCCCCTGCTGG
GAGTGGGGCTGAACACGACAATTCTGACGCCCAATGGGAATGAAGACACC
ACAGCTGATTTCTTCCTGACCACTATGCCCACTGACTCCCTCAGTGTTTC
CACTCTGCCCCTCCCAGAGGTTCAGTGTTTTGTGTTCAATGTCGAGTACA
TGAATTGCACTTGGAACAGCAGCTCTGAGCCCCAGCCTACCAACCTCACT
CTGCATTATTGGTACAAGAACTCGGATAATGATAAAGTCCAGAAGTGCAG
CCACTATCTATTCTCTGAAGAAATCACTTCTGGCTGTCAGTTGCAAAAAA
AGGAGATCCACCTCTACCAAACATTTGTTGTTCAGCTCCAGGACCCACGG
GAACCCAGGAGACAGGCCACACAGATGCTAAAACTGCAGAATCTGGTGAT
CCCCTGGGCTCCAGAGAACCTAACACTTCACAAACTGAGTGAATCCCAGC
TAGAACTGAACTGGAACAACAGATTCTTGAACCACTGTTTGGAGCACTTG
GTGCAGTACCGGACTGACTGGGACCACAGCTGGACTGAACAATCAGTGGA
TTATAGACATAAGTTCTCCTTGCCTAGTGTGGATGGGCAGAAACGCTACA
CGTTTCGTGTTCGGAGCCGCTTTAACCCACTCTGTGGAAGTGCTCAGCAT
TGGAGTGAATGGAGCCACCCAATCCACTGGGGGAGCAATACTTCAAAAGA
GAATCCTTTCCTGTTTGCATTGGAAGCCGTGGTTATCTCTGTTGGCTCCA
TGGGATTGATTATCAGCCTTCTCTGTGTGTATTTCTGGCTGGAACGGACG
ATGCCCCGAATTCCCACCCTGAAGAACCTAGAGGATCTTGTTACTGAATA
CCACGGGAACTTTTCGGCCTGGAGTGGTGTGTCTAAGGGACTGGCTGAGA
GTCTGCAGCCAGACTACAGTGAACGACTCTGCCTCGTCAGTGAGATTCCC
CCAAAAGGAGGGGCCCTTGGGGAGGGGCCTGGGGCCTCCCCATGCAACCA
GCATAGCCCCTACTGGGCCCCCCCATGTTACACCCTAAAGCCTGAAACCT
GAACCCCAATCCTCTGACAGAAGAACCCCAGGGTCCTGTAGCCCTAAGTG
GTACTAACTTTCCTTCATTCAACCCACCTGCGTCTCATACTCACCTCACC
CCACTGTGGCTGATTTGGAATTTTGTGCCCCCATGTAAGCACCCCTTCAT
TTGGCATTCCCCACTTGAGAATTACCCTTTTGCCCCGAACATGTTTTTCT
TCTCCCTCAGTCTGGCCCTTCCTTTTCGCAGGATTCTTCCTCCCTCCCTC
TTTCCCTCCCTTCCTCTTTCCATCTACCCTCCGATTGTTCCTGAACCGAT
GAGAAATAAAGTTTCTGTTGATAATCATCAAAAAAAAAAAAAAAAAAAAA
AAAAAAAAAA,

which includes an open reading frame that encodes a common-γ chain receptor (CD132) polypeptide having the amino acid sequence:

(SEQ ID NO: 11)
MLKPSLPFTSLLFLQLPLLGVGLNTTILTPNGNEDTTADFFLTTMPTDSL
SVSTLPLPEVQCFVFNVEYMNCTWNSSSEPQPTNLTLHYWYKNSDNDKVQ
KCSHYLFSEEITSGCQLQKKEIHLYQTFVVQLQDPREPRRQATQMLKLQN
LVIPWAPENLTLHKLSESQLELNWNNRFLNHCLEHLVQYRTDWDHSWTEQ
SVDYRHKFSLPSVDGQKRYTFRVRSRFNPLCGSAQHWSEWSHPIHWGSNT
SKENPFLFALEAVVISVGSMGLIISLLCVYFWLERTMPRIPTLKNLEDLV
TEYHGNFSAWSGVSKGLAESLQPDYSERLCLVSEIPPKGGALGEGPGASP
CNQHSPYWAPPCYTLKPET.

Variants, including alternative splice variants, mutants, and alternative isoforms of SEQ ID NOS:10 and 11 are also known in the art. See, for example, UniProtKB accession number: P31785 (IL2RG_HUMAN), which is specifically incorporated by reference in its entirety.

B. Methods of Detecting Biomarkers

The methods disclosed herein include detecting the expression level of one or more the biomarkers disclosed herein, in a subject or a biological sample obtained from the subject, and comparing them to a control.

1. Biological Samples

The disclosed methods of detection typically include detecting the expression level of one or more biomarkers in the cells of a biological sample obtain from a subject. The subject is typically a subject with cancer and the cells are typically cancer cells. The biological sample can include a single cancer cell, or preferable includes multiple cancer cells. The types of cancer that can be assayed and treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors can be classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

In some embodiments, the cancer or tumor is a type that has been associated with expression or over expression of B7-H1, such as breast cancer, colon cancer, esophageal cancer, gastric cancer, glioma, leukemia, lung cancer, melanoma, multiple myeloma, ovarian cancer, pancreatic cancer, renal cell carcinoma, and urothelial cancer.

In some embodiments, the biological sample includes cancer cells obtained from a tumor. In some embodiments, the biological sample includes cancer cells that are not obtained from a tumor. For example, in some embodiments, the cancer cells are circulating cancer cells. The biological sample can include other components or cells that are not cancer cells. For example, the sample can include non-cancerous cells, tissue, etc. In preferred embodiments, the biological sample includes cancer cells that isolated or separated away from normal tissue. In some embodiments, the biological sample is obtained from a cancerous tissue or organ.

A biological sample can be obtained from the subject using a variety of methods that are known in the art. In some embodiments, the sample is a tissue biopsy, for example a punch biopsy. The sample should be handled in accordance with the method of detection that will be employed. In some embodiments, a biological sample that is of tissue or cellular origin can be solubilized in a lysis buffer optionally containing one or more of a chaotropic agent, detergent, reducing agent, buffer, and salts. The conditions for handling biological samples that are analyzed for mRNA level may be different than the conditions for handling biological samples that are analyzed for protein level, and such conditions are known in the art. If the sample is a blood sample that include clotting factors (e.g., a whole blood sample), the preparation may include an anti-coagulant.

The sample can be concentrated, or diluted with a suitable diluent before the sample is analyzed. The sample can be frozen, fresh, fixed (e.g. formalin fixed), centrifuged, and/or embedded (e.g. paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation.

2. Methods of Detection

The detection of mRNA, polypeptides and proteins in a biological sample obtained from a subject is made possible by a number of conventional methods that are known in the art. The methods can be cell-based or cell-free assays.

For example, mRNA levels can be determined using assays, including, but not limited to, quantitative polymerase chain reaction (qPCR), also called real-time polymerase chain reaction, reverse transcription PCR (RT-PCR), reverse transcription real-time PCR (RT-qPCR), transcriptome analysis using next-generation sequencing, array hybridization analysis, digital PCR, Northern analysis, dot-blot, in situ hybridization, and RNase protection assay. In a preferred embodiment, the method includes detecting the level of HIF1-Alpha mRNA, KDR mRNA, CXCL13 mRNA, IL7R, or a combination thereof in mRNA isolated from cancer cells of the subject. In some embodiments, a probe for detecting HIF1-Alpha mRNA, KDR mRNA, CXCL13 mRNA, or IL7R is designed to hybridize with the nucleic acid sequence of SEQ ID NO:1, SEQ ID NO:3, SEQ ID NO:5, or SEQ ID NOS: 7, respectively.

In some embodiments, the assay include PCR, (e.g., qPCR, RT-PCR, RT-qPCR, etc.). Such PCR assays are well known in the art. For example, in some embodiments, a method for detecting mRNA from a VEGF isoform of interest in a biological sample includes producing cDNA from the sample by reverse transcription using at least one primer; amplifying the cDNA so produced; and detecting the presence of the amplified cDNA. In addition, such methods can include one or more steps that allow one to determine the levels of mRNA in a biological sample (e.g., by simultaneously examining the levels a comparative control mRNA sequence of a “housekeeping” gene such as an actin family member). Optionally, the sequence of the amplified cDNA can be determined. Northern blot analysis is a conventional technique well known in the art and is described, for example, in Sambrook, et al., Molecular Cloning, a Laboratory Manual, third edition, Cold Spring Harbor Press, NY (2000) 11803-2500.

In some embodiments, the biological sample contains a low quantity of cells, or is a single cell. Methods of amplifying cDNA and analyzing mRNA expression levels in low quantities of cells (e.g., 1,000 to 10 cells) and single cells, are reviewed and discussed in Pan and Weissman, Proc Natl Acad Sci USA, 8; 110(2):594-9 (2013). The methods can include, for example, semirandom primed PCR and phi29-based cDNA amplification steps.

Protein expression can be detected using routine methods, such as immunodetection methods including radioimmunoassays, ligand binding assays, mass spectroscopy, or high performance liquid chromatography (HPLC). In a preferred embodiment, the method includes detecting the level of a HIF1-Alpha protein or polypeptide, a KDR protein or polypeptide, a CXCL13 protein or polypeptide, a IL7R protein or polypeptide, or a combination thereof in protein isolated from cancer cells of the subject. In some embodiments, the protein detected includes the amino acid sequence of SEQ ID NO:2, SEQ ID NO:4, SEQ ID NO:6, or SEQ ID NOS:8 or 9, respectively or a fragment, homologue, alternative isoform, or variant thereof.

Some methods include immunoassays whereby polypeptides of the biomarker are detected by their interaction with a biomarker specific antibody. For example, if the biomarker is a HIF1-Alpha protein or polypeptide, a KDR protein or polypeptide, a CXCL13 protein or polypeptide, or a IL7R protein or polypeptide, the antibody or antibodies used in the assay is specific for HIF1-Alpha protein or polypeptide, KDR protein or polypeptide, CXCL13 protein or polypeptide, or IL7R protein or polypeptide, respectively. The biomarker can be detected in either a qualitative or quantitative manner. Exemplary immunoassays that can be used for the detection of biomarker polypeptides and proteins include, but are not limited to, radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP).

It will be appreciated that some immunoassays, for example ELISAs, can require two different biomarker specific antibodies or ligands (e.g., a capture ligand or antibody, and a detection ligand or antibody). In certain embodiments, the protein biomarker is captured with a ligand or antibody on a surface and the protein biomarker is labeled with an enzyme. In one example, a detection antibody conjugated to biotin or streptavidin—to create a biotin-streptavidin linkage to an enzyme that contains biotin or streptavidin. A signal is generated by the conversion of the enzyme substrate into a colored molecule and the intensity of the color of the solution is quantified by measuring the absorbance with a light sensor. Contemplated assays may utilize chromogenic reporters and substrates that produce an observable color change to indicate the presence of the protein biomarker. Fluorogenic, electrochemiluminescent, and real-time PCR reporters are also contemplated to create quantifiable signals.

Some assays optionally including fixing one or more antibodies to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe, substrate or a ProteinChip® array.

Flow cytometry is a laser based technique that may be employed in counting, sorting, and detecting protein biomarkers by suspending particles in a stream of fluid and passing them by an electronic detection apparatus. A flow cytometer has the ability to discriminate different particles on the basis of color. Differential dyeing of particles with different dyes, emitting in two or more different wavelengths allows the particle to be distinguished. Multiplexed analysis, such as FLOWMETRIX™ is discussed in Fulton, et al., Clinical Chemistry, 43(9):1749-1756 (1997) and can allow one to perform multiple discrete assays in a single tube with the same sample at the same time.

In some specific embodiments, the biomarker level(s) are measured using Luminex xMAP technology. Luminex xMAP is frequently compared to the traditional ELISA technique, which is limited by its ability to measure only a single analyte. The differences between ELISA and Luminex xMAP technology center mainly on the capture antibody support. Unlike with traditional ELISA, Luminex xMAP capture antibodies are covalently attached to a bead surface, effectively allowing for a greater surface area as well as a matrix or free solution/liquid environment to react with the analytes. The suspended beads allow for assay flexibility in a singleplex or multiplex format.

Commercially available formats that include Luminex xMAP technology includes, for example, BIO-PLEX® multiplex immunoassay system which permits the multiplexing of up to 100 different assays within a single sample. This technique involves 100 distinctly colored bead sets created by the use of two fluorescent dyes at distinct ratios. These beads can be further conjugated with a reagent specific to a particular bioassay. The reagents may include antigens, antibodies, oligonucleotides, enzyme substrates, or receptors. The technology enables multiplex immunoassays in which one antibody to a specific analyte is attached to a set of beads with the same color, and the second antibody to the analyte is attached to a fluorescent reporter dye label. The use of different colored beads enables the simultaneous multiplex detection of many other analytes in the same sample. A dual detection flow cytometer can be used to sort out the different assays by bead colors in one channel and determine the analyte concentration by measuring the reporter dye fluorescence in another channel.

In some specific embodiments, the biomarker(s) levels are measured using Quanterix's SIMOA™ technology. SIMOA™ technology (named for single molecule array) is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not. The digital nature of the technique allows an average of 1000× sensitivity increase over conventional assays with CVs <10%. Commercially available SIMOA™ technology platforms offers multiplexing options up to a 10-plex on a variety of analyte panels, and assays can be automated.

Typical protocols for evaluating the status of genes and gene products are found, for example in Ausubel et al. (eds.), Current Protocols In Molecular Biology (1995), Units 2 (Northern Blotting), 4 (Southern Blotting), 15 (Immunoblotting) and 18 (PCR Analysis).

Multiplexing experiments can generate large amounts of data. Therefore, in some embodiments, a computer system is utilized to automate and control data collection settings, organization, and interpretation.

3. Controls

The methods disclosed herein typically include comparing the level of the biomarker(s) detected in a sample obtained from the subject to a control or reference value. The Example below shows that HIF1-alpha and KDR have reduced expression and CXCL13 and IL7R have increased expression in cancer cells that are more responsive to immunotherapies including blockade of PD-1 signaling compared to cancer cells that are less responsive to immunotherapies including blockade of PD-1 signaling. Accordingly, in a preferred embodiment, the protein or mRNA level(s) of HIF1-alpha, KDR, CXCL13, IL7R, or a combination thereof in a biological test sample are compared to reference value(s) obtained for the same biomarker(s) from subject(s) that have or had cancer that is known to be responsive to immunotherapies including blockade of PD-1 signaling; subject(s) that have or had cancer that is known not to be responsive to immunotherapies including blockade of PD-1 signaling; or a combination thereof.

As used herein, a subject is “responsive” to an immunotherapy including blockade of PD-1 signaling if an immunotherapy including blockade of PD-1 signaling slows cancer or tumor growth, prevents cancer or tumor growth, or reduces one or more symptoms of the cancer or tumor, for example, tumor burden, after or following treatment with the immunotherapy. Therefore, in a preferred embodiment, the subject is responsive, if the immunotherapy reduces tumor burden during or after treatment. In some embodiments, a subject is responsive to the immunotherapy if the tumor or cancer goes into remission or is eradicated. Therefore, a “responder reference value” is a reference value obtained from one or more subjects that was responsive to an immunotherapy including blockade of PD-1 signaling.

As used herein, a subject is “non-responsive” to an immunotherapy including blockade of PD-1 signaling if an immunotherapy including blockade of PD-1 signaling does not slow cancer or tumor growth, does not prevent cancer or tumor growth, or does not reduce one or more symptoms of the cancer or tumor, for example, tumor burden, after or following treatment with the immunotherapy. Therefore, in a preferred embodiment, the subject is non-responsive, if tumor burden is increased during or after treatment. In some embodiments, a subject is non-responsive to the immunotherapy if the tumor or cancer expands, spreads, or metastasizes, or if one or more symptoms of the cancer worsen during or after treatment. Therefore, a “non-responder reference value” is a reference value obtained from one or more subjects that was non-responsive to an immunotherapy including blockade of PD-1 signaling.

It will be appreciated that the samples for preparing reference values are typically obtained from a reference subject prior to treatment with the immunotherapy. The reference value is later determined to be a responder reference value or non-responder reference value depending on whether or not the treatment was determined to be effective. Therefore, a method for preparing a reference valve can include, obtaining a sample from a reference subject prior to immunotherapy, determining the level of HIF1-alpha, KDR, CXCL13, IL7R, or a combination thereof in the sample to obtain reference values, administering to reference subject an immunotherapy, and categorizing the reference sample as responsive if the subject response to the immunotherapy, or non-responsive if the subject does not respond to the immunotherapy.

The duration of treatment with an immunotherapy for determining if a reference subject is responsive or non-responsive to the therapy can vary depending on conditions that are known in the art, including the age of the subject, the condition of his/her disease, the type of cancer being treated, and the specific treatment being employed.

The reference value can be obtained from a single subject, or a pool or average of two or more subjects. Preferably, the subject or subjects from which the reference value was obtained has the same type of cancer (e.g. breast prostate, etc.) as the test subject, however, this is not necessary. Preferably, the reference value(s) was obtained from the same tissue or cells as the test sample (e.g., epithelial cells, etc.), however, this is not necessary. Preferably the test sample is assayed using the same testing platform (e.g., analysis of mRNA by RT-PCT, analysis of protein by immunoassay, etc.) as was used to obtain the reference value(s). For example, in some embodiments, the reference value has been predetermined in the disease entity to which the patient belongs. The comparison between test values and references values can be an absolute quantification (e.g., comparison of numerical values), or relative (e.g., qualitatively comparison of color intensity, etc.).

In certain embodiments the reference level for responsive and non-responsive subjects is set to any percentage between 25% and 75% of the overall distribution of the values in a disease entity (e.g., type of cancer) investigated. In other embodiments the reference level is set to the median, tertiles or quartiles as determined from the overall distribution of the values in a disease entity investigated. In one embodiment the reference level is set to the median value as determined from the overall distribution of the values in a disease entity investigated. In preferred embodiments, the difference between a responder reference value and a non-responder reference value for a particular biomarker is statically significant.

In some embodiments, reference subjects are categorized by their level of responsiveness to treatment. For example, subjects can be categorized as “tumor eradication”, “tumor remission”, “reduced tumor burden”, “no tumor growth” etc., and used to establish a range of references levels of the biomarkers that correlate with the likelihood that an immunotherapy including PD-1 blockade of PD-1 signaling will be effective for treating a particular test subject.

### III. Methods of Using Biomarkers

The biomarkers can be used in a variety of diagnostic, prognostic, selection and treatment methods. For example, methods of identifying a subject who may benefit from treatment with an immunotherapy including blockade of PD-1 signaling are disclosed. Such methods typically include, determining the protein or mRNA expression level of the biomarker HIF1-alpha, KDR, CXCL13, IL7R, or any combination thereof in a sample obtained from the subject and comparing the level(s) of the biomarker(s) to a non-responder reference value, a responder reference value, or a combination thereof, wherein

(i) a level of HIF1-alpha in the sample obtained from the subject decreased compared to a non-responder reference value;

(ii) a level of HIF1-alpha in the sample obtained from the subject substantially the same or decreased compared a responder reference value;

(iii) a level of KDR in the sample obtained from the subject decreased compared to a non-responder reference value;

(iv) a level of KDR in the sample obtained from the subject substantially the same or decreased compared a responder reference value;

(v) a level of CXCL13 in the sample obtained from the subject increased compared to a non-responder reference value;

(vi) a level of CXCL13 in the sample obtained from the subject substantially the same or increased compared a responder reference value;

(vii) a level of IL7R in the sample obtained from the subject increased compared to a non-responder reference value;

(viii) a level of IL7R in the sample obtained from the subject substantially the same or increased compared a responder reference value; or

(ix) any combination thereof

indicates that the patient may benefit from treatment with the immunotherapy. Such subjects can be selected for treatment with the immunotherapy. In preferred embodiments, at least one, preferably two, three or all four of (i), (iii), (v), and (vii) are true. In more preferred embodiments, at least one, preferably two, three or all four of (ii), (iv), (vi), and (viii) are true.

Methods of predicting responsiveness of a subject suffering from cancer to treatment with an immunotherapy including blockade of PD-1 signaling are also provided. Such methods typically include, determining the protein or mRNA expression level of the biomarker HIF1-alpha, KDR, CXCL13, IL7R, or any combination thereof in a sample obtained from the subject and comparing the level(s) of the biomarker(s) to a non-responder reference value, a responder reference value, or a combination thereof, wherein

(i) a level of HIF1-alpha in the sample obtained from the subject decreased compared to a non-responder reference value;

(ii) a level of HIF1-alpha in the sample obtained from the subject substantially the same or decreased compared a responder reference value;

(iii) a level of KDR in the sample obtained from the subject decreased compared to a non-responder reference value;

(iv) a level of KDR in the sample obtained from the subject substantially the same or decreased compared a responder reference value;

(v) a level of CXCL13 in the sample obtained from the subject increased compared to a non-responder reference value;

(vi) a level of CXCL13 in the sample obtained from the subject substantially the same or increased compared a responder reference value;

(vii) a level of IL7R in the sample obtained from the subject increased compared to a non-responder reference value;

(viii) a level of IL7R in the sample obtained from the subject substantially the same or increased compared a responder reference value; or

(ix) any combination thereof

indicates that the subject will be responsive to treatment with the immunotherapy. Such subjects can be selected for treatment with the immunotherapy. In preferred embodiments, at least one, preferably two, three or all four of (i), (iii), (v), and (vii) are true. In more preferred embodiments, at least one, preferably two, three or all four of (ii), (iv), (vi), and (viii) are true.

Methods of determining the likelihood that a subject with cancer will exhibit benefit from an immunotherapy including blockade of PD-1 signaling are also provided. Such methods typically include, determining the protein or mRNA expression level of the biomarker HIF1-alpha, KDR, CXCL13, IL7R, or any combination thereof in a sample obtained from the subject and comparing the level(s) of the biomarker(s) to a non-responder reference value, a responder reference value, or a combination thereof, wherein

(i) a level of HIF1-alpha in the sample obtained from the subject decreased compared to a non-responder reference value;

(ii) a level of HIF1-alpha in the sample obtained from the subject substantially the same or decreased compared a responder reference value;

(iii) a level of KDR in the sample obtained from the subject decreased compared to a non-responder reference value;

(iv) a level of KDR in the sample obtained from the subject substantially the same or decreased compared a responder reference value;

(v) a level of CXCL13 in the sample obtained from the subject increased compared to a non-responder reference value;

(vi) a level of CXCL13 in the sample obtained from the subject substantially the same or increased compared a responder reference value;

(vii) a level of IL7R in the sample obtained from the subject increased compared to a non-responder reference value;

(viii) a level of IL7R in the sample obtained from the subject substantially the same or increased compared a responder reference value; or

(ix) any combination thereof

indicates the subject will exhibit benefit from the immunotherapy. Such subjects can be selected for treatment with the immunotherapy. In preferred embodiments, at least one, preferably two, three or all four of (i), (iii), (v), and (vii) are true. In more preferred embodiments, at least one, preferably two, three or all four of (ii), (iv), (vi), and (viii) are true.

In some embodiments, the level of biomarker(s) in test samples are compared to a range of responsive references values to determine the therapeutic index of a particular immunotherapy for a particular cancer. For example, a method of determining the therapeutic index of an immunotherapy can include determining the protein or mRNA expression level of the biomarker HIF1-alpha, KDR, CXCL13, IL7R, or any combination thereof in a test sample obtained from the subject and comparing the level(s) of the biomarker(s) to a series of responder reference values for the biomarker(s) that correlate with therapeutic index to determine the therapeutic index of the immunotherapy for the subject.

The disclosed methods can also be used to optimize therapeutic efficacy.

Methods of determining that a subject is not likely to be responsive to, or not likely to benefit from an immunotherapy including blocking of PD-1 signaling are also disclosed. Such methods typically include determining the protein or mRNA expression level of the biomarker HIF1-alpha, KDR, CXCL13, IL7R, or any combination thereof in a sample obtained from the subject and comparing the level(s) of the biomarker(s) to a non-responder reference value, a responder reference value, or a combination thereof, wherein

(i) a level of HIF1-alpha in the sample obtained from the subject substantial the same as or increased compared to a non-responder reference value;

(ii) a level of HIF1-alpha in the sample obtained from the subject increased compared a responder reference value;

(iii) a level of KDR in the sample obtained from the subject substantial the same as or increased compared to a non-responder reference value;

(iv) a level of KDR in the sample obtained from the subject increased compared a responder reference value;

(v) a level of CXCL13 in the sample obtained from the subject substantially the same or decreased compared to a non-responder reference value;

(vi) a level of CXCL13 in the sample obtained from the subject decreased compared a responder reference value;

(vii) a level of IL7R in the sample obtained from the subject substantially the same or decreased compared to a non-responder reference value;

(viii) a level of IL7R in the sample obtained from the subject decreased compared a responder reference value; or

(ix) any combination thereof

indicates the subject will not be responsive to, or not exhibit benefit from the immunotherapy. Such subjects can be selected for an alternative cancer therapy. In preferred embodiments, at least one, preferably two, three or all four of (ii), (iv), (vi), and (viii) are true. In more preferred embodiments, at least one, preferably two, three or all four of (i), (iii), (v), and (vii) are true.

Any of the disclosed methods of diagnosis, prognosis, selection, or determination of efficacy can be coupled to a step or subsequent method of treating the subject.

### IV. Immunotherapies Including Blockade of PD-1 Signaling

It has been shown that both B7-H1 and B7-DC bind to PD-1 (Freeman, et al., J. Exp. Med., 192:1027-1034 (2000)), a distant member of the CD28 family with an immunoreceptor tyrosine-based inhibitory motif (ITIM) in its cytoplasmic domain (Ishida, et al., EMBO J., 11:3887-3895 (1992)). PD-1, a member of the CD28 family of receptors, is inducibly expressed on activated T cells, B cells, natural killer (NK) cells, monocytes, DC, and macrophages (Keir, et al., Curr. Opin. Immunol. 19:309-314 (2007)).

The primary result of PD-1 ligation by its ligands is to inhibit signaling downstream of the T cell Receptor (TCR). Therefore, signal transduction via PD-1 usually provides a suppressive or inhibitory signal to the T cell that results in decreased T cell proliferation or other reduction in T cell activation. B7-H1 is the predominant PD-1 ligand causing inhibitory signal transduction in T cells. Immunotherapies can address the problem of undesired T cell inhibition by providing agents that bind to PD-1 and thus prevent inhibitory signal transduction, or else bind to ligands of PD-1 such as B7-H1, thereby preventing the ligand from binding to PD-1 to deliver an inhibitory signal. In either case, T cell responses, such as T cell proliferation or activation, are stimulated.

B7-H1 is the predominant PD-1 ligand, likely due to its broader distribution and higher expression levels. PD-1 inhibition occurs only when PD-1 and TCR are ligated in close proximity to each other, in the context of the immune synapse.

B7-H1 is also over expressed in many cancers (including breast cancer, colon cancer, esophageal cancer, gastric cancer, glioma, leukemia, lung cancer, melanoma, multiple myeloma, ovarian cancer, pancreatic cancer, renal cell carcinoma, and urothelial cancer), and has been linked to poor prognosis. B7-H1 is expressed by many tumor cell lines, especially following stimulation with interferon gamma (IFN-γ), and is also upregulated on tumor infiltrating myeloid derived suppressor cells (MDSC). For example, PD-1 is up-regulated on tumor specific CD8 T cells and is associated with functional impairment, anergy, exhaustion, and apoptosis. PD-1 upregulation has also been associated with dysfunctional and/or suppressive phenotypes on additional cell types, such as regulatory T cells (Treg) and natural killer T (NKT) cells.

As discussed above, the methods disclosed herein are typically directed to immunotherapies that including blockade of PD-1 signaling. Immunotherapies that include blocking PD-1 inhibitory signaling are well known in the art and typically include administering a subject an antagonist of PD-1 or an antagonist of a ligand of PD-1. The immunotherapies can include administering the subject one or more additional agents. In preferred embodiments, the immunotherapy includes administering the subject a potentiating agent, a vaccine, or combination thereof.

Immunotherapeutic compositions including an antagonist of PD-1 signaling and methods of use thereof are discussed in U.S. Pat. No. 8,114,845, Mkrtichyan, et al., Eur J Immunol., 41(10):2977-86 (2011) and Mkrtichyan, et al., J Immunol., 189(5):2338-47 (2012), each of which are specifically incorporated by reference herein in their entireties.

It will be appreciated that the immunotherapeutic compositions and methods of use disclosed below are applicable to all of the methods disclosed herein, including methods of detection, diagnosis, prognosis, selection and treatment. For example, in some embodiments, an immunotherapy disclosed below is the immunotherapy used to establish a reference value; in some embodiments, an immunotherapy below is used to treat a subject that has been identified as responsive to immunotherapy, etc.

A. Compositions for Immunotherapy

1. Antagonists of PD-1 Inhibitory Signaling

Exemplary antagonists of PD-1 signaling are discussed above, and include, but are not limited to, compounds that bind to and block PD-1 receptors on T cells without triggering inhibitory signal transduction, compounds that bind to PD-1 ligands to prevent their binding to PD-1, compounds that do both and compounds that prevent expression of genes that encode either PD-1 or natural ligands of PD-1. The blockade can be complete, or incomplete. Therefore, in some embodiments, “blockade of PD-1 signaling,” refers to complete inhibition of PD-1 signaling in a cell. In some embodiments, “blockade of PD-1 signaling” refers to reducing or decreasing PD-1 signaling.

Compositions containing antagonists of PD-1 receptors are provided and include compounds or agents that either bind to or block a ligand of PD-1 to interfere with or inhibit the binding of the ligand to the PD-1 receptor, or bind directly to and block the PD-1 receptor without inducing inhibitory signal transduction through the PD-1 receptor. In another embodiment, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

PD-1 signaling requires binding to a PD-1 ligand (such as B7-H1 or B7-DC) in close proximity to a peptide antigen presented by major histocompatibility complex (MHC) (see, for example, Freeman Proc. Natl. Acad. Sci. U.S.A 105:10275-10276 (2008)). Therefore, proteins, antibodies or small molecules that prevent co-ligation of PD-1 and TCR on the T cell membrane are also useful PD-1 antagonists.

Exemplary PD-1 antagonists include, but are not limited to B7-DC polypeptides, including homologs and variants of these, as well as active fragments of any of the foregoing, and fusion proteins that incorporate any of these. In a preferred embodiment, the fusion protein comprises the soluble portion of B7-DC coupled to the Fc portion of an antibody, such as human IgG, and does not incorporate all or part of the transmembrane portion of human B7-DC. The PD-1 receptor antagonists can also be small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

PD-1 antagonists include fragments of the B7-DC protein incorporating the ECD. Alternatively, the fragments of B7-DC include part of the extracellular domain that comprise the an IgV or IgV-like domain, preferably amino acids 20-221, more preferably 20-121, that are sufficient to bind to the PD-1 receptor to interfere with, or prevent, or otherwise reduce inhibitory signal transduction through the PD-1 receptor. In a preferred embodiment the B7-DC fragment competes with B7-H1 for binding to PD-1 receptors.

PD-1 antagonists include B7-DC fusion proteins, particularly B7-DC-Ig fusion proteins that include the extracellular domain of B7-DC. In one non-limiting example, human B7-DC fusion proteins contain amino acids 20-221 of human B7-DC fused to amino acids 245-476 of human IgG1 (AAA02914). The signal peptides for B7-DC fusion proteins include the endogenous signal peptides or any other signal peptide that facilitates secretion of the fusion protein from a host or during recombinant expression. In another embodiment, the B7-DC domain of the fusion protein includes only the IgV domain. Some embodiments include the hinge and Fc domain of an IgG antibody, such IgG1, with none of the variable region present. Other embodiments include use of the hinge and Fc region of IgG2 or IgG4, especially having an N297Q or other mutation that reduces effector function.

In another embodiment, the antagonist of PD-1 signaling is or includes a fragment of a mammalian B7-H1, preferably from mouse or primate, preferably human, wherein the fragment binds to and blocks PD-1 but does not result in inhibitory signal transduction through PD-1. The fragment can be at least 10, or at least 20, or at least 30, or at least 40, or at least 50, or at least 60, or at least 70, or at least 80, or at least 90, or at least 100 contiguous amino acids in length. In other embodiments, the fragment can be of variable length so long as it has the function of binding to PD-1 but does not produce inhibitory signal transduction that results in reduced T cell proliferation.

Such B7-H1 fragments also find use as part of the first polypeptide portion of fusion proteins. B7-H1-Ig proteins are described in WO/2001/014557 (pub. 1 Mar. 2001) and in WO/2002/079499 (pub. 10 Oct. 2002).

Other useful polypeptides include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., Crystal structure of the complex between programmed death-1 (PD-1) and its ligand PD-L2, PNAS, Vol. 105, pp. 10483-10488 (29 Jul. 2008)). B7-1 or soluble fragments thereof, which can bind to the B7-H1 ligand and prevent binding to the endogenous PD-1 receptor, thereby preventing inhibitory signal transduction, are also useful.

Because B7-1 and fragments thereof can also bind to B7-H1 and send inhibitory transduction to T cells through B7-H1, blocking of this interaction can also reduce inhibitory signal transduction that occurs through B7-H1. Compounds for use in the immunotherapies herein therefore include those molecules that block this type of interaction. Such molecules have been disclosed in Butte et al (2007), supra, and include anti-B7-H1 antibodies with dual-specificity that block either the B7-H1:B7-1 and B7-H1:PD-1 interaction as well as antibodies exhibiting mono-specificity that block the PD-L1:B7-1 interaction. Compounds that block this interaction by blocking B7-1 are also useful, and include anti-B7-1 antibodies.

Other PD-1 antagonists include antibodies that bind to PD-1 or ligands of PD-1, and other antibodies. Anti-PD-1 antibodies include, but are not limited to, those described in the following publications:
PCT/IL03/00425 (Hardy et al., WO/2003/099196)
PCT/JP2006/309606 (Korman et al., WO/2006/121168)
PCT/US2008/008925 (Li et al., WO/2009/014708)
PCT/JP03/08420 (Honjo et al., WO/2004/004771)
PCT/JP04/00549 (Honjo et al., WO/2004/072286)
PCT/IB2003/006304 (Collins et al., WO/2004/056875)
PCT/US2007/088851 (Ahmed et al., WO/2008/083174)
PCT/US2006/026046 (Korman et al., WO/2007/005874)
PCT/US2008/084923 (Terrett et al., WO/2009/073533)
Berger et al., Clin. Cancer Res., Vol. 14, pp. 30443051 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of about 3 mg/kg.

Anti-B7-H1 antibodies include, but are not limited to, those described in the following publications:
PCT/US06/022423 (WO/2006/133396, pub. 14 Dec. 2006)
PCT/US07/088851 (WO/2008/083174, pub. 10 Jul. 2008)
US 2006/0110383 (pub. 25 May 2006)

A specific example of an anti-B7-H1 antibody is MDX-1105 (WO/2007/005874, published 11 Jan. 2007)), a human anti-B7-H1 antibody.

For anti-B7-DC antibodies see U.S. Pat. Nos. 7,411,051, 7,052,694, and 7,390,888, and U.S. Published Application No. 20060099203

In some embodiments, the antibodies are bi-specific and including an antibody that binds to the PD-1 receptor bridged to an antibody that binds to a ligand of PD-1, such as B7-H1. In a preferred embodiment, the PD-1 binding portion reduces or inhibits signal transduction through the PD-1 receptor.

The PD-1 antagonist can also be small molecule antagonist. The term “small molecule” refers to small organic compounds having a molecular weight of more than 100 and less than about 2,500 daltons, preferably between 100 and 2000, more preferably between about 100 and about 1250, more preferably between about 100 and about 1000, more preferably between about 100 and about 750, more preferably between about 200 and about 500 daltons. The small molecules often include cyclical carbon or heterocyclic structures and/or aromatic or polyaromatic structures substituted with one or more functional groups. The small molecule antagonists reduce or interfere with PD-1 receptor signal transduction by binding to ligands of PD-1 such as B7-H1 and B7-DC and preventing the ligand from interacting with PD-1 or by binding directly to and blocking the PD-1 receptor without triggering signal transduction through the PD-1 receptor.

PD-1 antagonists can also be anti-sense nucleic acids, both DNA and RNA, as well as siRNA molecules. Such anti-sense molecules prevent expression of PD-1 on T cells as well as production of T cell ligands, such as B7-H1, PD-L1 and PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are Readily Taken up by cells that express PD-1 as well as ligands of PD-1 and reduce expression of these receptors and ligands to achieve a decrease in inhibitory signal transduction in T cells, thereby activating T cells.

2. Potentiating Agents

The activity of the PD-1 antagonist can be increased by the presence of a potentiating agent.

In the preferred embodiment, the potentiating agent is cyclophosphamide or an analog thereof. Cyclophosphamide (CTX, Cytoxan®, or Neosar®) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety).

Ifosfamide (MITOXANA) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis.

Oxazaphosphorines derivatives have been designed and evaluated with an attempt to improve the selectivity and response with reduced host toxicity (Liang J, Huang M, Duan W, Yu X Q, Zhou S. Design of new oxazaphosphorine anticancer drugs. Curr Pharm Des. 2007; 13(9):963-78. Review). These include mafosfamide (NSC 345842), glufosfamide (D19575, beta-D-glucosylisophosphoramide mustard), S-(−)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

In some embodiments, the potentiating agent is an IL-10 antagonist such as an anti-IL-10 antibody. Anti-IL-10 antibodies are known in the art and have been administered to subjects to treat autoimmune disease. See, for example, Asadullah, et al., Pharmacological Reviews, 55(2) 241-269 (2003), Llorente, et al., Arthritis Rheum., 43(8):1790-800 (2000), and U.S. Published Application Nos. 2014/0004126 and 2012/0321617. In a particular embodiments, the antibody is B-N10.

In some embodiments, the potentiating agent is an agent that reduces activity and/or number of regulatory T lymphocytes (T-regs), such as Sunitinib (SUTENT®), anti-TGFβ, or Imatinib (GLEEVAC®). Other useful potentiating agents include mitosis inhibitors, such as paclitaxol, aromatase inhibitors (e.g. Letrozole) and angiogenesis inhibitors (VEGF inhibitors e.g. Avastin, VEGF-Trap) (see, for example, Li et al., Clin Cancer Res., 12(22):6808-16 (2006), anthracyclines, oxaliplatin, doxorubicin, TLR4 antagonists, and IL-18 antagonists.

3. Vaccines

Vaccines include antigens, and optionally other adjuvants and targeting molecules.

a. Antigens Typically, the vaccines disclosed herein include an antigen expressed by or characteristic of the cancer being treated. In the most preferred embodiments, the antigen is a tumor or cancer specific antigen. Antigens can be peptides, proteins, polysaccharides, saccharides, lipids, nucleic acids, or combinations thereof. The antigen can be derived from a virus, bacterium, parasite, protozoan, fungus, histoplasma, tissue or transformed cell and can be a whole cell or immunogenic component thereof, e.g., cell wall components or molecular components thereof.

Suitable antigens are known in the art and are available from commercial, government and scientific sources. In one embodiment, the antigens are whole inactivated or attenuated organisms. These organisms may be infectious organisms, such as viruses, particular is the cancer is associated with a viral infection. The antigens may be tumor cells or cells infected with a virus. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

Many tumor antigens are known in the art and include, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Tumor antigens, such as BCG, may also be used as an immunostimulant to adjuvant.

b. Adjuvants

Optionally, the vaccines may include an adjuvant. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g., lysolecithin, pluronic polyols, polyanions, peptides, oil emulsions, keyhole limpet hemocyanin, and dinitrophenol).

Adjuvants may also include immunomodulators such as cytokines, interleukins (e.g. IL-1, IL-2, IL-4, IL-5, IL-6, IL-7, IL-12, etc.), interferons (e.g., interferon-gamma), macrophage colony stimulating factor, and tumor necrosis factor. Other co-stimulatory molecules, including other polypeptides of the B7 family, may also be administered. Such proteinaceous adjuvants may be provided as the full-length polypeptide or an active fragment thereof, or in the form of DNA, such as plasmid DNA.

In some embodiments, the vaccine includes an antigen, Incomplete Freund's adjuvant, or GM-CSF, or a combination thereof. In a particular embodiment, the vaccine includes an antigen, Incomplete Freund's adjuvant, and GM-CSF.

Vaccine compositions (as discussed below) may further incorporate additional substances to stabilize pH, or to function as adjuvants, wetting agents, or emulsifying agents, which can serve to improve the effectiveness of the vaccine.

Vaccines are generally formulated for parenteral administration and are injected either subcutaneously or intramuscularly. Such vaccines can also be formulated as suppositories or for oral administration, using methods known in the art, or for administration through nasal or respiratory routes.

B. Methods of Immunotherapy

The immunotherapy methods disclosed herein typically include administering a subject in need thereof an antagonist of PD-1 signaling, and optionally a potentiating agent such as cyclophosphamide or anti-IL-10 antibody, and optionally a vaccine. Therapeutically effective amounts of PD-1 antagonists optionally in combination with a potentiating agent, and/or a vaccine can cause an immune response to be activated or sustained. The combination therapies are useful in enhancing T cell responses, through increased T cell activity, increased T cell proliferation and reduced T cell inhibitory signals, and can be used in treating (or even preventing) diseases such as cancer.

The selected dosage depends upon the desired therapeutic effect, on the route of administration, and on the duration of the treatment desired. Generally dosage levels of 0.001 to 50 mg/kg of body weight daily are administered to mammals. Preferably, said dose is 1 to 50 mg/kg, more preferably 1 to 40 mg/kg, or even 1 to 30 mg/kg, with a dose of 2 to 20 mg/kg being also a preferred dose. Examples of other dosages include 2 to 15 mg/kg, or 2 to 10 mg/kg or even 3 to 5 mg/kg, with a dose of about 4 mg/kg being a specific example.

For treatment regimens using a potentiating agent and an antibody, antibody dosages are commonly in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred (plus a suitable dose of cyclophosphamide or other potentiating agent given up to about 24 hours before the antibody).

In general, by way of example only, dosage forms based on body weight for any of the signal transduction antagonists useful in the disclosed methods include doses in the range of 5-300 mg/kg, or 5-290 mg/kg, or 5-280 mg/kg, or 5-270 mg/kg, or 5-260 mg/kg, or 5-250 mg/kg, or 5-240 mg/kg, or 5-230 mg/kg, or 5-220 mg/kg, or 5-210 mg/kg, or 20 to 180 mg/kg, or 30 to 170 mg/kg, or 40 to 160 mg/kg, or 50 to 150 mg/kg, or 60 to 140 mg/kg, or 70 to 130 mg/kg, or 80 to 120 mg/kg, or 90 to 110 mg/kg, or 95 to 105 mg/kg, with doses of 3 mg/kg, 5 mg/kg, 7 mg/kg, 10 mg/kg, 15 mg/kg, 20 mg/kg, 25 mg/kg, 30 mg/kg, 50 mg/kg and 100 mg/kg being specific examples of preferred doses.

Such doses may be repeated. The dose will, of course, be correlated with the identity of the mammal receiving said dose. Doses in the above-recited mg/kg ranges are convenient for mammals, including rodents, such as mice and rats, and primates, especially humans, with doses of about 5 mg/kg, about 10 mg/kg and about 15 mg/kg being especially preferred for treating humans.

The potentiating agent, for example cyclophosphamide, is typically administered in non-toxic dose that varies depending on the agent and the animal. In specific embodiments, the potentiating agent is administered by any suitable means of administration, including parenteral or oral, the former including system administration, such as intravenous. For example, a potentiating agent like cyclophosphamide is normally administered orally. Such administration may be at any convenient dosage, depending on the potentiating agent. The dosage in each case may be based on body weight or may be administered as a unit dosage.

While CTX itself is nontoxic, some of its metabolites are cytotoxic alkylating agents that induce DNA crosslinking and, at higher doses, strand breaks. Many cells are resistant to CTX because they express high levels of the detoxifying enzyme aldehyde dehydrogenase (ALDH). CTX targets proliferating lymphocytes, as lymphocytes (but not hematopoietic stem cells) express only low levels of ALDH, and cycling cells are most sensitive to DNA alkylation agents.

Low doses of CTX (<200 mg/kg) can have immune stimulatory effects, including stimulation of anti-tumor immune responses in humans and mouse models of cancer (Brode & Cooke Crit Rev. Immunol. 28:109-126 (2008)). These low doses are sub-therapeutic and do not have a direct anti-tumor activity. In contrast, high doses of CTX inhibit the anti-tumor response. Several mechanisms may explain the role of CTX in potentiation of anti-tumor immune response: (a) depletion of CD4+CD25+FoxP3+ Treg (and specifically proliferating Treg, which may be especially suppressive), (b) depletion of B lymphocytes; (c) induction of nitric oxide (NO), resulting in suppression of tumor cell growth; (d) mobilization and expansion of CD11b+Gr-1+MDSC. These primary effects have numerous secondary effects; for example following Treg depletion macrophages produce more IFN-γ and less IL-10. CTX has also been shown to induce type I IFN expression and promote homeostatic proliferation of lymphocytes.

Treg depletion is most often cited as the mechanism by which CTX potentiates the anti-tumor immune response. This conclusion is based in part by the results of adoptive transfer experiments. In the AB1-HA tumor model, CTX treatment at Day 9 gives a 75% cure rate. Transfer of purified Treg at Day 12 almost completely inhibited the CTX response (van der Most et al. Cancer Immunol. Immunother. 58:1219-1228 (2009). A similar result was observed in the HHD2 tumor model: adoptive transfer of CD4+CD25+ Treg after CTX pretreatment eliminated therapeutic response to vaccine (Taieb, J. J. Immunol. 176:2722-2729 (2006)).

Numerous human clinical trials have demonstrated that low dose CTX is a safe, well-tolerated, and effective agent for promoting anti-tumor immune responses (Bas, & Mastrangelo Cancer Immunol. Immunother. 47:1-12 (1998)).

The optimal dose for CTX to potentiate an anti-tumor immune response, is one that lowers overall T cell counts by lowering Treg levels below the normal range but is subtherapeutic (see Machiels, et al. Cancer Res. 61:3689-3697 (2001)).

In human clinical trials where CTX has been used as an immunopotentiating agent, a dose of 300 mg/m² has usually been used. For an average male (6 ft, 170 pound (78 kg) with a body surface area of 1.98 m²), 300 mg/m² is 8 mg/kg, or 624 mg of total protein. In mouse models of cancer, efficacy has been seen at doses ranging from 15-150 mg/kg, which relates to 0.45-4.5 mg of total protein in a 30 g mouse (Machiels et al. Cancer Res. 61:3689-3697 (2001), Hengst et al Cancer Res. 41:2163-2167 (1981), Hengst Cancer Res. 40:2135-2141 (1980)).

For larger mammals, such as a primate, preferably human, patient, such mg/m² doses may be used but unit doses administered over a finite time interval may be preferred. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention. The same regimen may be applied for the other potentiating agents recited herein.

Administrations of potentiating agent and/or vaccine can occur before, after, or simultaneously with administration of a PD-1 antagonist. Alternatively, administration of one or more doses of a PD-1 antagonist can be temporally staggered with the administration of potentiating agent and/or vaccine to form a uniform or non-uniform course of treatment. For example, in a particular embodiment, one or more doses of potentiating agent are administered, followed by one or more doses of a PD-1 binding compound, followed by one or more doses of potentiating agent, all according to whatever schedule is selected or desired by the researcher or clinician administering said agents.

In other specific embodiments, the treatment regimen includes multiple administrations of one or more PD-1 antagonists. In some embodiments, such multiple administrations of PD-1 antagonists are in conjunction with multiple administrations of the same or different potentiating agents and/or the same or different vaccines.

In a particular embodiment, a treatment regime includes administering the subject a potentiating agent 1, 2, 3, 4, 5, 6, 7, 8, 10, 12, 13, 14, 15, 16, 17, 18, 19, 20, 21, 22, 23, 24, or 30 hours, or 1, 1.5, 2, or 3 days before administration of the PD-1 antagonist. In some embodiments, the vaccine is co-administered with the PD-1 antagonist.

PD-1 antagonist, cyclophosphamide, and vaccine can be administered in the same or separate pharmaceutical compositions. Therefore, the combination therapies can include administration of the active agents together in the same admixture, or in separate admixtures. A pharmaceutical composition can include two, three, or more active agents. The pharmaceutical compositions can be formulated as a pharmaceutical dosage unit, referred to as a unit dosage form. Pharmaceutical compositions useful herein typically contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition).

Pharmaceutical compositions can be administered by enteral (oral), parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The methods can include administering the PD-1 antagonist and the potentiating agent and/or the vaccine by separate and different routes (e.g. topically).

### V. Other Cancer Treatments

In some of the methods disclosed herein, the subject may be identified as non-responsive to an immunotherapy that includes blockade of PD-1 signaling. In such cases, the subject may be treated for cancer using different approach. For example, such subjects may be treated other immunotherapies that do not include blockade of PD-1 signaling, including adoptive T-cell therapy, dendritic cell therapy, monoclonal antibody therapy, interferon therapy, immune checkpoint therapy, etc. Other traditional approaches to treating cancer can also be employed. For example, subjects can be treated with chemotherapy, radiation therapy, surgery, hormone therapy, photodynamic therapy, anti-angiogenesis therapy, etc.

### VI. Devices and Kits

Devices and kits for detection of biomarkers are also disclosed. Using the methods and systems of the present disclosure, several types of markers can be detected. The marker being detected may indicate whether subject be responsive to immunotherapy. The marker being detected may be a nucleic acid (or polynucleotide) or a protein (or polypeptide). The marker being detected can determine the format of the test (i.e., assay, strip, etc.), and/or the type of biomolecular recognition element (e.g. antibodies, antigens, etc.) being used to detect the marker. The marker being detected may be a single marker or a combination of markers. The marker being detected may be specific to one condition or multiple conditions.

There may be provided a test or support surface used for performing a test for detecting the presence of a selected marker(s). The test or support surface may be coated with/hold the selected detection antibodies, etc. specific to the marker(s) being detected.

The device or kit typically includes reagents and/or apparatus that can be used to carry out the test. Some kits include an apparatus that includes a support surface for the detection of the marker. The surface, can be, for example a surface on which the selected detection antibodies, etc. can be coated/held for detection of the selected marker(s). In some embodiments, the test or support surface may be part of an assay having one or more containers (or wells). The test or support surface may be the inner surface of a well or container. The inner surface of one or more wells or containers may be coated with the detection antibody specific to the marker(s) being detected.

Any appropriate assay or ELISA (sandwich, indirect, competitive, reverse, etc.) can be provided as part of the kit or device. For example, the kits or device can provided a polystyrene microplate, having wells/containers with inner surfaces capable of being coated with antibody. These inner surfaces may or may not be treated with substances known in the art to promote or enhance coating. For example the surface can be a maxisorp, POLYSORP, medisorp, MINISORP or COVALINK surface. Each well or container may be white or opaque to allow for easier visualization of any color, or any visually detectable change, occurring in or on the well or container. It will be appreciated that the size, surface area, total and/or working volumes, appearance, and/or color/visual parameters and/or qualities can be modified as desired within the scope of the present disclosure.

In some embodiments, the test or support surface may be part of a vial (or container or well), a test strip, a chromatography substrate, a gene chip, a SNAP test, or any other diagnostic test or test system used for detecting markers. The test or support surface may be made of paper, plastic, glass, metal, etc. and take several forms such as paddle, beads, wells, electrodes, etc.

In some embodiments, non-specific adsorption to the test surfaces coated with the BRE (e.g. the detection antibody), such as the coated well/container of an assay, may be minimized by blocking the test surface with a blocking agent. The blocking agent may be one or more proteins, sugars and/or polymers such as bovine serum albumin, gelatin, polyethylene glycol, sucrose, etc.

The kit or device can include an appropriate biomolecular recognition element (BRE), for detection of the biomarker. In some embodiments, the test surface is coated with the BRE (e.g., the detection antibody). The coated surface, such as the coated well/container of an assay, may be coated with a preserving (or stabilizing) agent to preserve the activity of the test surface. Test surfaces coated with the BRE and the blocking agent may also be coated with the preserving agent. The preserving agent may allow the test surfaces coated with the preserving agent, and the BRE and/or blocking agent, to be stored for an extended period of time before use. Test surfaces coated with the preserving agent, and the BRE and/or blocking agent, may maintain immunological activity for several months compared to if no preserving agent is employed (where immunological activity of a test surface coated with the BRE and/or a blocking agent may continually decline over time).

In some embodiments, the marker being detected, when present in increased or increasing amounts, may indicate a positive/reactive result. In some embodiments, the marker being detected, when absent or present in decreased or decreasing amounts, may indicate a positive/reactive result.

To detect if a marker is present in a sample, a signal from the sample may be compared against the signals of a high standard and a low standard which can be included with the kit or device. A qualitative/visual signal may be generated or visualized of the sample and test standards for making the comparison. The visual indicator may visualize or generate a signal of the sample and standards having a magnitude corresponding to the level of the marker present. The visual indicator may visualize or generate a signal for the first standard consistent with a first level of marker. The visual indicator may visualize a signal for the second standard consistent with a second level of marker.

For example, the visual indicator may visualize for the high standard a signal consistent with a level, such as the minimum level, of the biomarker in a subject with the disease or disorder. The visual indicator may visualize for the low standard a signal consistent with a level, such as the maximum level, of the biomarker in a subject without the disease or disorder. The magnitude of the signal from the biological test sample generated by the visual indicator may be compared against the standards to determine the diagnosis.

Generating the visually detectable signal can be accomplished in several ways. Any visual indicator, including any dye, chromogen, substance, substrate, or solution capable of producing a qualitative indication or visually detectable change may be utilized and included with the kit or device. The generated signal may be visually detectable with or without special equipment. For example, the signal may be a color change, or the generation of a color change along a spectrum, that is visible without special equipment. In some embodiments, it is possible to detect changes in light absorbance visually, with non-specialized light detection equipment, or specialized equipment (e.g., Spectrophotometer). In some embodiments, the signal may be detected by measuring a change in a physical or chemical property of the substrate being tested based on the presence of a label, such as an enzyme label. Types of enzyme-labeled signals known to the art include: light absorbance, light emission, fluorescence, electrochemical signal, pH, etc.

The kits and devices can include instructions for use.

In some embodiments, the kit or device is used to assaying a cell sample, such as those discussed above.

Devices that can assist in carrying out the methods disclosed herein are also provided. Included are devices that assist in taking or analyzing biopsies. For example, core needle biopsy instruments, vacuum-assisted biopsy systems, etc.

### Example

### Materials and Methods

Mice were implanted with TC-1 tumor cells, and once tumors were palpable, fine needle biopsies were taken from all tumors. After that, mice were either treated with a single low dose of cyclophosphamide (CTX)/vaccine/anti-PD-1 antibody or anti-IL-10/vaccine/anti-PD-1 antibody. A background of the immunotherapeutic strategy is discussed in Mkrtichyan et al, Eur J Immunol., 41(10):2977-86 (2011) and Mkrtichyan, et al, J Immunol., 189(5):2338-47 (2012), both of which are specifically incorporated by reference herein in their entireties.

Treated animals were categorized as “responders” and “non-responders”. The fine needle biopsies that were taken before treatment was analyzed using Affymetrix gene array (over 28,000 coding transcripts) to identify differential gene expression between “responders” and “non-responders”.

### Results

Both treatment with the combination of CTX/vaccine/anti-PD-1 antibody and the combination of anti-IL-10/vaccine/anti-PD-1 antibody resulted in complete regression of established tumors in 50% of animals. For the conclusion of the treatment, animals were categorized as “responders” and “non-responders”. The fine needle biopsies that were taken before treatment was analyzed using Affymetrix gene array (over 28,000 coding transcripts) to identify differential gene expression between “responders” and “non-responders”.

Four genes with greatest expression differences between “responders” versus “non-responders” were identified. Expression of two of these genes (HIF-alpha and KDR) was significantly lower in biopsies from “responder” mice compared to “non-responders”. Another two genes (CXCL13 and IL7R) expressed at significantly higher level in biopsies from “responders” versus “non-responders”.

Thus these four genes can serve as biomarkers that would predict the success (e.g., therapeutic potency) of treatments based on combination of vaccines, anti-PD-1 antibodies, and low dose of CPM or anti-IL-10 antibody.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of identifying a subject who may benefit from treatment with an immunotherapy that includes blockade of PD-1 signaling comprising determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a non-responder reference value, a responder reference value, or a combination thereof, wherein
(i) a level of HIF1-alpha in the sample obtained from the subject decreased compared to a non-responder reference value;
(ii) a level of HIF1-alpha in the sample obtained from the subject substantially the same or decreased compared a responder reference value;
(iii) a level of KDR in the sample obtained from the subject decreased compared to a non-responder reference value;
(iv) a level of KDR in the sample obtained from the subject substantially the same or decreased compared a responder reference value;
(v) a level of CXCL13 in the sample obtained from the subject increased compared to a non-responder reference value;
(vi) a level of CXCL13 in the sample obtained from the subject substantially the same or increased compared a responder reference value;
(vii) a level of IL7R in the sample obtained from the subject increased compared to a non-responder reference value;
(viii) a level of IL7R in the sample obtained from the subject substantially the same or increased compared a responder reference value; or
(ix) any combination thereof
indicates that the patient may benefit from treatment with the immunotherapy.

2. A method of predicting responsiveness of a subject suffering from cancer to treatment with an immunotherapy including blockade of PD-1 signaling comprising determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a non-responder reference value, a responder reference value, or a combination thereof, wherein
(i) a level of HIF1-alpha in the sample obtained from the subject decreased compared to a non-responder reference value;
(ii) a level of HIF1-alpha in the sample obtained from the subject substantially the same or decreased compared a responder reference value;
(iii) a level of KDR in the sample obtained from the subject decreased compared to a non-responder reference value;
(iv) a level of KDR in the sample obtained from the subject substantially the same or decreased compared a responder reference value;
(v) a level of CXCL13 in the sample obtained from the subject increased compared to a non-responder reference value;
(vi) a level of CXCL13 in the sample obtained from the subject substantially the same or increased compared a responder reference value;
(vii) a level of IL7R in the sample obtained from the subject increased compared to a non-responder reference value;
(viii) a level of IL7R in the sample obtained from the subject substantially the same or increased compared a responder reference value; or
(ix) any combination thereof
indicates that the subject will be responsive to treatment with the immunotherapy.

3. The method of claim 1, wherein two, three or all four of (i), (iii), (v), and (vii) are true.

4. The method of any of claim 1, wherein one, two, three or all four of (ii), (iv), (vi), and (viii) are true.

5. A method of determining the therapeutic efficacy of an immunotherapy comprising blockade of PD-1 signaling comprising determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a range of responsive references values to determine the therapeutic index of a particular immunotherapy for a particular cancer, wherein each of the responder reference values correlates with therapeutic efficacy on the immunotherapy.

6. A method of determining that a subject is not likely to be responsive to, or not likely to benefit from an immunotherapy comprising blocking of PD-1 signaling comprising determining the protein or mRNA expression level of a biomarker selected from the group consisting of HIF1-alpha, KDR, CXCL13, IL7R, and any combination thereof in a cancer cell sample obtained from the subject and comparing the level(s) of the biomarker(s) to a non-responder reference value, a responder reference value, or a combination thereof, wherein
(i) a level of HIF1-alpha in the sample obtained from the subject substantial the same as or increased compared to a non-responder reference value;
(ii) a level of HIF1-alpha in the sample obtained from the subject increased compared a responder reference value;
(iii) a level of KDR in the sample obtained from the subject substantial the same as or increased compared to a non-responder reference value;
(iv) a level of KDR in the sample obtained from the subject increased compared a responder reference value;
(v) a level of CXCL13 in the sample obtained from the subject substantially the same or decreased compared to a non-responder reference value;
(vi) a level of CXCL13 in the sample obtained from the subject decreased compared a responder reference value;
(vii) a level of IL7R in the sample obtained from the subject substantially the same or decreased compared to a non-responder reference value;
(viii) a level of IL7R in the sample obtained from the subject decreased compared a responder reference value; or
(ix) any combination thereof
indicates the subject will not be responsive to, or will not exhibit benefit from the immunotherapy.

7. The method of claim 6 wherein two, three or all four of (ii), (iv), (vi), and (viii) are true.

8. The method of claim 6 wherein at least one, preferably two, three or all four of (i), (iii), (v), and (vii) are true.

9. The method of claim 1 wherein the mRNA or protein levels of at least two of HIF1-alpha, KDR, CXCL13, and IL7R in the cancer cell sample are determined.

10. The method of claim 1 wherein the mRNA or protein levels of at least three of HIF1-alpha, KDR, CXCL13, and IL7R in the cancer cell sample are determined.

11. The method of claim 1 wherein the mRNA or protein levels of at least two of HIF1-alpha, KDR, CXCL13, and IL7R in the cancer cell sample are determined.

12. The method of claim 1 wherein the mRNA level of the biomarker is determined.

13. The method claim 12 wherein the mRNA level of the biomarker is determined by quantitative polymerase chain reaction (qPCR), reverse transcription PCR (RT-PCR), reverse transcription real-time PCR (RT-qPCR), transcriptome analysis using next-generation sequencing, array hybridization analysis, digital PCR, Northern analysis, dot-blot, in situ hybridization, and RNase protection assay.

14. The method of claim 13 wherein the protein level of the biomarker is determined.

15. The method of claim 14 wherein the protein level is determined by immunoassay, ligand binding assay, mass spectroscopy, or high performance liquid chromatography (HPLC).

16. The method of claim 15 wherein the immunoassay is selected from the group consisting of radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP)

17. The method of claim 1 wherein the responder reference value is prepared by determining the protein or mRNA expression level of the corresponding biomarker(s) in a reference cancer cell sample obtained from a reference subject prior to treatment with the immunotherapy and wherein the immunotherapy was effective to slow cancer or tumor growth, prevent cancer or tumor growth, or reduce one or more symptoms of the cancer or tumor in the reference subject.

18. The method of claim 1 wherein the non-responder reference value is prepared by determining the protein or mRNA expression level of the corresponding biomarker(s) in a reference cancer cell sample obtained from a reference subject prior to treatment with the immunotherapy and wherein the immunotherapy was not effective to slow cancer or tumor growth, not effective to prevent cancer or tumor growth, or not effective to reduce one or more symptoms of the cancer or tumor in the reference subject.

19. The method of claim 1 further comprising administering the subject the immunotherapy if the subject was determined to be responsive or to benefit from the immunotherapy.

20. The method of claim 1 further comprising administering to the subject chemotherapy, radiation therapy, surgery, hormone therapy, photodynamic therapy, or anti-angiogenesis therapy if the subject was determined not to be responsive or not to benefit from the immunotherapy.


SEQUENCE LISTING (11 sequences)

SEQ ID NO: 1 - DNA, 4082 bp, Homo sapiens
gcgcgcgccggcctgggcaggcgagcgggcgcgctcccgccccctctcccctccccgcgcgcccgagcgcgcctccgcccttgcccgccccctgacgctgcctcagctcctcagtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggcaaaaaaaaaaaaaaaaaaaaaaa

SEQ ID NO: 2 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 3 - DNA, 6055 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 4 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 5 - DNA, 1219 bp, Homo sapiens
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 6 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 7 - DNA, 4617 bp, Homo sapiens
atctaagcttctctgtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaagaaagaaaggaaaataaaaaatgatagttgccattattaggatttaatatatatccagtgctttgcaagtgctctgcgcaccttgtctcactccatcctgacaataatcctgggaggtgtgtgcaattactacgactactctcttttttatagatcattaaattcagaactaaggagttaagtaacttgtccaagttgttcacacagtgaagggaggggccaagatatgatggctgggagtctaattgcagttccctgagccatgtgcctttctcttcactgaggactgccccattcttgagtgccaaacgtcactagtaacagggtgtgcctagataatttatgatccaaactgagtcagtttggaaagtgaaagggaaacttacatataatccctccgggacaatgagcaaaaactaggactgtccccagacaaatgtgaacatacatatcatcacttaaattaaaatggctatgagaaagaaagagggggagaaacagtcttgcgggtgtgaagtcccatgaccagccatgtcaaaagaaggtaaagaagtcaagaaaaagccatgaagcccatttggtttcatttttctgaaaataggctcaagagggaataaattagaaactcacaatttctcttgtttgttaccaagacagtgattctcttgctgctaccacccaactgcatccgtccatgatctcagaggaaactgtcgctgaccctggacatgggtacgtttgacgagtgagaggaggcatgacccctcccatgtgtatagacactaccccaacctaaattcatccctaaattgtcccaagttctccagcaatagaggctgccacaaacttcagggagaaagagttacaagtacatgcaatgagtgaactgactgtggctacaatcttgaagatatacggaagagacgtattattaatgcttgacatatatcatcttgcctttcttggtctagactgacttctaatgactaactcaaagtcaaggcaactgagtaatgtcagctcagcaaagtgcagcaaacccatctcccacaggcctccaaaccctggctgttcacagaaccacaaagggcagatgctgcacagaaaactagagaaggggtcataggttcatggttttgtttgagatttgttgctactgtttttctgttttgaattttcttctttgttctgtttttactttatttagggggactaggtgtttctgatattttagttttcttgtttgttttgttttgtgttgtctgtgaatggggttttaactgtggatgaatggaccttatctgttggcttaaaggactggtaagatcagaccatcttattcttcaggtgaatgttttactttccaaagtgctctcctctgcaccagcagtaataaatacaatgccataatcccttaggtttgcctagtgcttttgcaattttcaaagcacttccataagcattccttccacctccttgataggcatttatggaaagcctgctacatgtcaatcatactgttaggcacaggggacctaaagacacataaaaggatggcattctgcctcataaattgcaaaacctaatgaaagtgactgcttggtaaacaaattattattatattataaaatgctataaaagagccatattgaaagtgccctgttggagacagggcaaatgccacaaaaatgatgtaaatttacatggaggaaaagtagaatctgcctggtttgtaggcagcagaagacatttttcatcagtgggcaggtgttctttaccttttgtagaaatgggagtcaagtctcaaataggaggctccacaaaatctcatgccaggtctctgataccttattcacagaagttctttgaagtatttattgttattttctttgacttatgggaaaactgggacacaggaagacaggtaaattacccaacctcacacgttaagtcagaactgggagccataattttgtatccctggtataaatagacaatctcttgaagaaatgaagagatgaccatagaaaaacatcgagatatctccagctctaaaatcctttgtttcaatgttgtttggcatatgttatctttggaatttagtgtctgagcctctgtctgttactgtagtatttaaaatgcatgtattataatcatataatcataactgctgttaattcttgattatatacctagggacaatgtgtaatgtaagattactaattggttctgcccaatctcctttcagattttattaggaaaaaaaaataaacctcctgatcggagacaatgtattaatcagaagtgtaaactgccagttctatatagcatgaaatgaaaagacagctaatttggtccaacaaacatgactgggtctagggcacccaggctgattcagctgatttcctaccagcctttgcctcttccttcaatgtggtttccatgggaatttgcttcagaaaagccaagtatgggctgttcagaggtgcacacctgcattttcttagctcttctagaggggctaagagacttggtacgggccaggaagaatatgtggcagagctcctggaaatgatgcagattaggtggcatttttgtcagctctgtggtttattgttgggactattctttaaaatatccattgttcactacagtgaagatctctgatttaaccgtgtactatccacatgcattacaaacatttcgcagagctgcttagtatataagcgtacaatgtatgtaataaccatctcatatttaattaaatggtatagaagaacaaaaaaaaaaaaaaaaaa

SEQ ID NO: 8 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 9 - protein, 459 aa, Homo sapiens [sequence omitted]

SEQ ID NO: 10 - DNA, 1560 bp, Homo sapiens
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaaaaaaaaaaaaaaaaaaaaaa

SEQ ID NO: 11 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET